(19) United States
(12) Patent Application Publication
(10) Pub. No.: US 2024/0327493 A1
(43) Pub. Date: Oct. 3, 2024

(54) PD-1 AGONIST

(71) Applicant: GEORGE MASON UNIVERSITY, Fairfax, VA (US)

(72) Inventors: Rachel Marie Carter, Fairfax, VA (US); Amanda Nicole Haymond Still, Fairfax, VA (US); Lance Allen Liotta, Fairfax, VA (US); Alessandra Luchini Kunkel, Fairfax, VA (US); Mikell Paige, Fairfax, VA (US); Anne Brown, Fairfax, VA (US); Amanda Sharp, Fairfax, VA (US)

(21) Appl. No.: 18/620,631

(22) Filed: Mar. 28, 2024

Related U.S. Application Data
(60) Provisional application No. 63/492,532, filed on Mar. 28, 2023.

(51) Int. Cl. C07K 14/705; A61K 38/00; A61P 37/06

(52) U.S. Cl. CPC C07K 14/70532; A61P 37/06; A61K 38/00

(57) ABSTRACT

Embodiments relate to peptides that binds with molecules of programmed death protein-1 (PD-1) to form a complex that modulate release of an inflammatory cytokine from a cell. Peptides are PD-1 agonists used to treat an inflammatory disease.

### CROSS-REFERENCE TO RELATED APPLICATIONS

This application claims the benefit of U.S. Patent Application No. 63/492,532, filed on Mar. 28, 2023, which is hereby incorporated by reference in its entirety.

### FIELD OF THE INVENTION

This invention relates to PD-1 agonist. The invention is more particularly concerned with peptide-based PD-1 agonist.

### SEQUENCE LISTING

The instant application contains a Sequence Listing which has been submitted electronically in XML format created using WIPO ST.26 sequence listing. XML format is hereby incorporated by reference in its entirety. Said XML format created on Mar. 28, 2024, is named GMUN-034-01US.xml and is 20.4 KB in size.

### BACKGROUND OF INVENTION

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra, Okazaki et al. (2002) Curr. Opin. Immunol 14:391779-82; Bennett et al. (2003) J Immunol 170:711-8).

PD-1 on the surface of some immune cells (most prominently T-cells), and PD-L1, a molecule on antigen presenting cells, can bind together to stimulate an immunosuppressive reaction whereby immune cells are prohibited from releasing inflammatory cytokines.

The PD-1/PD-L1 checkpoint pathway has been the target of immunotherapies due to its important role in the negative regulation of the immune system. PD-1 (programmed cell death protein 1) is a receptor mainly found on the surface of T cells, and when bound by its ligand, PD-L1, inhibits downstream signaling pathways that promote T cell activation1. In T cells, PD-1/PD-L1 signaling inhibits early TCR/CD28 signaling via dephosphorylation, leading to the inactivation of downstream pathways that normally promote T cell proliferation and release of pro-inflammatory cytokines.

Antagonists of the PD-1/PD-L1 complex have become successful anti-cancer treatments, particularly for cancers with high PD-L1 expression or liquid tumors.

Continued suppression via PD-1 signaling leads to T-cell exhaustion (1). As exhausted T-cells cannot effectively clear malignant cells, some cancers selectively upregulate PD-L1 to exploit this effect.

Cancer treatment via checkpoint therapies (antibodies targeting PD-1 or PD-L1 that function as antagonists of the complex) can lead to enhanced immune response and subsequent side effects (6, 7).

PD-1/PD-L1 axis disruption not only reduces beneficial immune signaling, such as in some cancers, but also promotes inappropriate immune signaling, leading to autoimmunity. Diseases which have been shown to incorporate an autoimmune component due to aberrant or deficient PD-1/PD-L1 immunosuppression include arthritis (2), diabetes (3), rheumatic diseases such as lupus (4), and chronic pain (5).

An agonist of PD-1 could be used as a “rescue therapy” to abrogate the flare-up of autoimmune disease side effects following PD-1 or PD-L1 targeting checkpoint therapies. Because of the incredible commercial potential of PD-1 agonists, a recent review has called such drugs “the future of autoimmune disease therapeutics” (8).

A number of different groups have focused on the development of PD-1/PD-L1 agonists. Primarily, these agonists have been antibodies, including targeting co-receptors of PD-L1 like CD80, leading to increased frec PD-L1 for PD-1 binding (9); targeting PD-1 at the non-PD-L1 binding site (10); or bi-specifically targeting PD-1 and other markers (11).

Development of agonists based on the sequence of PD-L1, such as reports of 40-amino acid mini-proteins designed to bind at the PD-1/PD-L1 interface, have also been reported by the Baker group (12).

While high-affinity therapeutics targeting PD-1 have been shown to inhibit PD-1 signaling, studies of soluble PD-L1 have shown conflicting results as to whether cell-free PD-L1 agonizes or antagonizes PD-1 signaling. Many studies have shown that the presence of soluble PD-L1 in cancer patients correlates to worse clinical outcomes due to activation of PD-1 signaling and subsequent T cell suppression (10-12). However, a recent study suggested that a splice variant of secreted PD-L1 could act as an antagonist for PD-1 (13). That is, development of high-affinity forms of PD-L1, which would be expected to act as agonists, were instead shown to function as antagonists (13).

It is therefore unclear precisely why some PD-1 binders act as agonists versus antagonists” especially when both bind to the PD-1/PD-L1 interface. Such activity could be a function of a yet undiscovered precise 3-D binding region, with subtle differences governing agonism vs antagonism, as well as a function of affinity, with higher affinity molecules reducing PD-1/PD-L1 signaling while lower affinity molecules (approaching the PD-1/PD-L1 complex affinity) increase signaling.

Further, mAbs targeting PD-1/PD-11 are expensive to produce due to the high cost of manufacturing and have been found to be cost-ineffective for patients, with patients paying up to $100,000/year for treatment.

Regulation of immune responses is central for the prevention of inflammatory and autoimmune disorders. While downregulation of the immune system can be achieved by way of immunosuppressive therapy, agents that generally suppress the immune system leave subjects susceptible to other disorders, including infections and cancers. A means for controlling the aberrant activation of an immune response is required. There is a long felt need of a PD-1 agonist that would stimulate the effects of natural PD-1/PD-L1 binding, leading to enhanced immune suppression. It would be able to provide answers to questions of agonism versus antagonism.

### SUMMARY OF INVENTION

The present invention addresses the question about agonism versus antagonism by the unexpected discovery of a specific small PD-L1 agonist peptide, and, in turn, its cognate 3-D binding site on the PD-1 immune cell receptor which offers a novel class of therapeutic targets.

Additionally, given the discrepancies associated with agonism and antagonism of PD-1, we wanted to determine if simple blockade of just the crystallographically determined binding region of PD-1 and PD-L1 (14) was sufficient to fully recapitulate the native PD-L1 signaling.

An embodiment relates to agonists of PD-1 molecules that would stimulate the effects of natural PD-1/PD-L1 binding, leading to enhanced immune suppression. Molecules with this kind of activity would thus represent a completely new class of drugs to treat autoimmunity related disease. Such PD-1 agonists could be used for patients with the PD-1/PD-L1 based autoimmune disorders.

In one embodiment, the composition comprises a variant peptide of SEQ ID NO. 5, wherein the variant peptide has at least 85% homologous to SEQ ID No. 5, wherein the variant peptide does not contain mutation at tyrosine 12 residue of SEQ ID NO. 5.

In an embodiment, the variant peptide thereof binds with molecules of programmed death protein-1 (PD-1) to form a complex that modulate release of an inflammatory cytokine from a cell. In an embodiment, the variant peptide is PD-1 agonist.

In an embodiment, the variant peptide comprises one or more amino acids of SEQ ID NO. 5 chemically modified.

In an embodiment, a chemical modification comprises methylation of an amino acid.

In an embodiment, the chemical modification is configured to increase serum stability of the variant peptide compared to SEQ ID NO. 5.

In an embodiment, the variant peptide has a serum stability of at least about 6 hours.

In an embodiment, the serum stability is in a range of 0 hours to 100 hours.

In an embodiment, the variant peptide degrades less than 50% in the serum for 50 hours.

In an embodiment, the inflammatory cytokine comprises IL-2, TNFα, and/or GM-CSF.

In an embodiment, the variant peptide decreases release of the inflammatory cytokine in a dose dependent manner.

In an embodiment, the variant peptide comprises cyclization of SEQ ID NO. 5.

In an embodiment, cyclization of the variant peptide is through the addition of residues in SEQ ID NO. 1 for formation of disulfide bonds.

In an embodiment, cyclization of the variant peptide is through the addition of residues in SEQ ID NO. 1 for formation of thioether bonds.

In one embodiment, the variant peptide comprises SEQ ID NO. 9, wherein the thioether bond is established between C-terminal cysteine residue and N-terminal A residue.

In an embodiment, the variant peptide comprises SEQ ID NO. 11, wherein K(N₃) is an azido-lysine residue, and the disulfide bond is established between the C-terminal cysteine residue and N-terminal residue.

In an embodiment, the variant peptide comprises SEQ ID NO. 5, wherein N-terminal and C-terminal residues form the disulfide bond to cyclize the peptide.

In an embodiment, the variant peptide has one or more residues truncated from N-terminus and/or C-terminus of SEQ ID NO. 5, such that the variant peptide is protected from acetylation or amidation.

In an embodiment, the variant peptide comprises a fluorine analog.

In an embodiment, the variant peptide has one or more amino acids of SEQ ID NO.1 or SEQ ID NO. 5 comprising branch forming compounds to increase stability of the variant peptide.

In an embodiment, the branch forming compounds comprises olefin terminated amino acids.

In an embodiment, the variant peptide comprises SEQ ID NO. 12.

In an embodiment, the variant peptide comprises SEQ ID NO. 13.

In an embodiment, arginine in SEQ ID NO. 1 is substituted with N-methyl arginine.

In an embodiment, the arginine towards C-terminal in SEQ ID NO. 1 is substituted with N-methyl arginine.

In an embodiment, the variant peptide does not contain mutation at 2, 4, or 11-14 residues of SEQ ID NO. 1

In an embodiment, the variant peptide comprises SEQ ID NO. 7 having additional hydrogen bond compared to SEQ ID NO. 1.

In an embodiment, SEQ ID NO. 5 has a serum stability from 0 hours to 100 hours.

In an embodiment, the composition has a therapeutic amount of the variant peptide.

In an embodiment, the composition is configured to treat an autoimmune disease in the subject.

In an embodiment, the therapeutic amount of the variant peptide is configured to decrease release of inflammatory cytokine in a cancer by about 25% to 95%.

In an embodiment, the therapeutic amount of the variant peptide is configured to decrease release of inflammatory cytokine in an autoimmune inflammatory disease by about 25% to 95%.

In an embodiment, the composition further comprises one or more pharmaceutical acceptable carriers.

In an embodiment, the variant peptide does not contain mutation at 2, 4, or 11-14 residues of SEQ ID NO. 1.

In an embodiment, a complex comprising a variant peptide comprising SEQ ID NO. 5 and PD-1, wherein the variant peptide is about 90% homologous to SEQ ID NO. 1, and the PD-1 has its native 3-D configuration.

In an embodiment, the variant peptide attaches to lysine78 molecule on PD-1.

In an embodiment, a method of treatment comprising taking a pharmaceutical composition comprising a peptide selected from SEQ ID NO. 1 to SEQ ID NO. 13 and a pharmaceutical salt, administering to a subject a therapeutic amount of the pharmaceutical composition, wherein the pharmaceutical composition is configured to treat an Immune-related disease.

In an embodiment, the pharmaceutical composition is configured to suppress release of an inflammatory cytokine.

In an embodiment, wherein the Immune-related disease comprises an autoimmune disease.

In an embodiment, the peptide comprises SEQ ID NO. 5.

In an embodiment, Immune-related disease comprises diabetes, arthritis, lupus, cancer or chronic pain.

In an embodiment, the inflammatory cytokine comprises IL-2, TNFα, or GM-CSF.

In an embodiment, the pharmaceutical composition decreases TNFα more compared to IL-2 in the same cancer.

In an embodiment, the pharmaceutical composition decreases TNFα more compared to GM-CSF in the same cancer.

In an embodiment, suppression of the inflammatory cytokine is about 10 to 99% compared to a second subject not treated with the pharmaceutical composition.

In an embodiment, suppression of the inflammatory cytokine is about 20 to 95% compared to a second subject not treated with the pharmaceutical composition.

In an embodiment, suppression of TNFα is more compared to rest of the inflammatory cytokine.

### BRIEF DESCRIPTION OF THE FIGURES

The accompanying drawings, which are included to provide further understanding of the present invention disclosed in the present disclosure and are incorporated in and constitute a part of this specification, illustrate aspects of the present invention and together with the description serve to explain the principles of the present invention. In the drawings:

FIG. 1 Structure of SEQ ID NO. 2 (MN 1.1), color coded according to the change in affinity as shown in Table 2.

FIG. 2 shows AlphaScreen validation demonstrates that mutation of Y7 is tolerated while Y12E eliminates PD-1/PD-L1 blocking activity. [MN 2.1 (SEQ ID NO. 3), MN_Null (SEQ ID NO. 14, MN 1.1 (SEQ ID NO. 2)]

FIG. 3 shows Design and sequence of MN1.1 highlighted in hPD-1/hPD-L1 4ZQK crystal structure (A). (B) PD-1 shown with only the MN1.1 region of PD-L1.

FIG. 4 shows stability studies indicating cyclization and N-methylation modifications significantly improve peptide half-life in serum; the dissociation constant measured via SPR for stabilized peptide MN1.4 shows similarities to wild-type PD-L1. (A) MALDI-TOF results of serum stability studies with MN1.1 (2,038 Da) and MN1.4 (1,820 Da). Time points were taken at 0 minutes, 15 minutes, 1 hour, and 6 hours. Peptide levels were measured via matrix-assisted laser desorption/ionization (MALDI)-time of flight (TOF) mass spectrometry using Angiotensin I as a loading control.

FIG. 5 shows Quantification of ESI-MS results for MN1.1 (SEQ ID NO. 1), MN1.2 (SEQ ID NO. 4), and MN1.4 (SEQ ID NO. 5) serum stability experiments. Timepoints were taken at 0, 6, 24, and 48 hours.

FIG. 6 shows Representative trial of SPR experiments with PD-1 and MN1.4.

FIG. 7 shows molecular dynamics simulations of MN1.1 on left. CD results with MN1.1 and MN1.2 shown on right.

FIG. 8 shows AlphaScreen results testing inhibition of complex formation by peptides optimized for affinity.

FIG. 9 shows AlphaScreen results testing inhibition of complex formation by peptides optimized for stability.

FIG. 10 shows MN1.4 functions as a PD-1 agonist, reducing secretion of inflammatory cytokines from human PBMCs in a co-culture model. A) MN1.4 decreased released IL-2 in a dose dependent manner in a co-culture model, in which cancer cells outnumbered PBMCs 10:1. B) To validate, the model was rerun with I cancer cell for every 10 PBMCs, which would be expected to reduce the potency of a PD-1 agonist. A new female donor was used as compared to panel A to ensure the effects observed were not donor specific. A dose-dependent reduction in IL-2 was observed. C) Identical experiment conducted as in Panel B, but samples were assayed for TNFα. A reduction in this cytokine was also observed following MN1.4 treatment. D) Controls including recombinant PD-L1 and MN-Null were compared to MN1.4. MN1.4 was shown to clicit the same reduction in TNFα as rPD-L1, while the null peptide did not show a significant reduction.

### DETAILED DESCRIPTION

### Definitions and General Techniques

For simplicity and clarity of illustration, the drawing figures illustrate the general manner of construction, and descriptions and details of well-known features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements but may include other elements not expressly listed or inherent to such process, method, system, article, device, or apparatus.

No element, act, or instruction used herein should be construed as critical or essential unless explicitly described as such. Also, as used herein, the articles “a” and “an” are intended to include items, and may be used interchangeably with “one or more.” Furthermore, as used herein, the term “set” is intended to include items (e.g., related items, unrelated items, a combination of related items, and unrelated items, etc.), and may be used interchangeably with “one or more.” Where only one item is intended, the term “one”, or similar language, is used. Also, as used herein, the terms “has,” “have,” “having,” or the like are intended to be open-ended terms. Further, the phrase “based on” is intended to mean “based, at least in part, on” unless explicitly stated otherwise.

The present invention may be embodied in other specific forms without departing from its spirit or characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

As defined herein, “approximately”, “substantially” or “about” can, in some embodiments, mean within plus or minus ten percent of the stated value. In other embodiments, “approximately” “substantially” or “about” can mean within plus or minus five percent of the stated value. In further embodiments, “approximately” “substantially” or “about” can mean within plus or minus three percent of the stated value. In yet other embodiments, “approximately” “substantially” or “about” can mean within plus or minus one percent of the stated value.

Unless otherwise defined herein, scientific and technical terms used in connection with the present invention shall have meanings that are commonly understood by those of ordinary skill in the art. Further, unless otherwise required by context, singular terms shall include pluralities and plural terms shall include the singular. Generally, nomenclatures used in connection with, and techniques of, health monitoring described herein are those well-known and commonly used in the art.

The methods and techniques of the present invention are generally performed according to conventional methods well known in the art and as described in various general and more specific references that are cited and discussed throughout the present specification unless otherwise indicated. The nomenclatures, procedures, and techniques of embodiments herein, and other related fields described herein, are those well-known and commonly used in the art.

The recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications, without departing from the spirit and scope of the invention. Also, the terminology and phrascology used is for the purpose of describing exemplary embodiments and should not be considered limiting. Thus, the present invention is to be accorded the widest scope encompassing numerous alternatives, modifications and equivalents consistent with the principles and features disclosed. For the purpose of clarity, details relating to technical material that are known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

Definitions of some terms used in the specification are as follows:

The term “PD-1” as used herein refers to human PD-1, and the terms immunostimulatory receptor, a membrane protein forming a complex of the immunostimulatory receptor, and a membrane protein located in the same immunological synapse with the immunostimulatory receptor are refer to those derived from human. Examples of the immunostimulatory receptor include T-cell receptors (TCRs), B-cell receptors (BCRs), cytokine receptors, LPS receptors, complement receptors, and Fc receptors. Examples of the membrane protein forming an immunostimulatory receptor complex include CD3 and CD79. Examples of the membrane protein located in the same immunological synapse with the immunostimulatory receptors include CD2 and CD19.

PD-1 has an extracellular region containing immunoglobulin superfamily domain, a transmembrane domain, and an intracellular region including an immunoreceptor tyrosine-based inhibitory motif (ITIM). PD-1 transmits a negative signal to immune cells, similar to CTLA4. PD-1 ligand proteins are expressed on the surface of antigen presenting cells, and other cell types; and can provide a costimulatory signal to immune cells or can transmit downmodulatory signals to immune cells, depending upon the protein to which they bind.

The term “PD-L1” as used herein is a Type I membrane protein of 272 amino acids (precursor=290 amino acids) comprising an extracellular portion (from about residues 19 to 238) that includes an IgV domain (from about residues 18 to 130), a transmembrane domain (from about residues 239 to 261 and a short intracellular tail (from about residues 262 to 290), as shown in US20170226182A1. The domains and residues of human PD-1 and PD-L1 involved in their interaction is described in for example Lin et al., Proc. Natl. Acad. Sci. 2008 105 (8): 3011-3016. For example: IgV domains of PD-1 (from about residues 35 to 145, and more particularly residues 64, 66, 68, 73-76, 78, 90, 122, 124, 126, 128, 130-132, 134 and 136) and PD-L1 (from about residues 18 to 130, and more particularly residues 19, 20, 26, 54, 56, 66, 113, 115, 117, and 121-125) are involved in the interaction. Similarly, the domains and residues of mouse PD-1 and PD-L1 involved in their interaction is described in for example Lazar-Molnar et al., Proc. Natl. Acad. Sci. 2008 105 (30): 10483-10488.

The term “PD-1 agonist” as used herein refers to an agent capable of inducing/triggering the PD-1 signalling pathway in a cell. It includes agents that binds to PD-1 in its native 3-D configuration on the surface of living cell and triggers an intracellular signal, such as a natural or synthetic PD-1 ligand.

The term, “complex” as used herein refers to attachment of the peptide with one or more molecules of the PD-1. The PD-1 may be in its native 3-D configuration. In an embodiment, the complex formed due to attachment of the peptide to a molecule of PD-1 may mimic binding action of PD-1 and PD-L1 with respect to that molecule. The attachment may due to formation of a covalent bond. In another case, the attachment may be due non-covalent formation.

The term, “stable” refers to the stability of a peptide, in a particular environment such as not limited to within a human body or in presence of human fluid such as but not limited to serum, at a particular pH and temperature for a particular time-period without leading to substantial degradation of the peptide. For example, degradation of the peptide is less than 10%, 20%, 30%, 40%, 50%, 60%, 70%, 80% or 90%. In an example: particular time period could be 15 mins, 30 mins, 60 mins, 1 hour, 2 hours, 10 hours, 20 hours, 30 hours, 40 hours, 50 hours, 60 hours, 70 hours, 80 hours, 90 hours, 100 hours, 120 hours, 150 hours, 200 hours or more. In another example, the time period could be selected from 1, 2, 4, 8, 10, 20, 30, 60, 90 days or more. In an example: pH could be 6, 6.5, 7, 7.5, 8. In an example: temperature could be 4° C., 10° C., 15° C., 20° C., 25° C., 30° C., 37° C., 45° C.

The terms “inhibit”, “inhibition, “suppress” and “suppression” in terms of an immune response includes the decrease, limitation or blockage of, for example, a particular action, function or interaction (e.g. suppression of production or secretion of inflammatory cytokine).

The term, “inflammatory cytokine” refers to cytokine (a signalling molecule) that is secreted from immune cells and certain other cell types that promote inflammation. Examples of inflammatory cytokines include but not limited to interleukin-1 (IL-1), IL-12, IL-18, tumor necrosis factor alpha (TNF-α), interferon gamma (IFNγ), and granulocyte-macrophage colony stimulating factor (GM-CSF).

Terms “polypeptide,” “protein” and “peptide” are used interchangeably to denote a sequence polymer of at least two amino acids covalently linked by an amide bond, regardless of length or post-translational modification (e.g., glycosylation, phosphorylation, lipidation, myristilation, ubiquitination, etc.). D- and L-amino acids, and mixtures of D- and L-amino acids are also included. In an embodiment, peptides disclosed herein include, for example, high affinity binding to human PD-1, which lacks substantial cross-reactivity with either human CD28, CTLA-4 or ICOS.

The term “variant” as used herein refers to polypeptide sequences that may include substitutions, variations, or deletions of one or more amino acids, so long as the modified polypeptide has substantially similar or enhanced activity or function compared to the unmodified polypeptide. For example: a peptide may be modified so that it at least maintains, if not improves, the ability to interact with and bind to a binding groove of a PD-1 molecule and suppresses the immune system. In an embodiment, the disclosure includes variant peptide of the SEQ ID NO. 1. In an embodiment, the disclosure includes variant peptide of SEQ ID NO. 5. Alternatively, the disclosure allows modification of amino acid sequence SEQ ID NO: 1 to SEQ ID NO. 14, such that the variant peptide has similar way of attaching to PD-1 and leading to immune suppression. In an embodiment, the variant has any of about 85%, 90%, 95% or 99%, 99.9% sequence similarity with the SEQ ID NO. 1 to SEQ ID NO. 14.

The term “homologous” as used herein refers to the degree of identity or similarity between sequences of two amino acid sequences, i.e. peptide or polypeptide sequences. The aforementioned “homology” is determined by comparing two sequences aligned under optimal conditions over the sequences to be compared. Such a sequence homology can be calculated by creating an alignment using, for example, the ClustalW algorithm. Commonly available sequence analysis software, more specifically, Vector NTI, GENETYX or other tools are provided by public databases. In an embodiment, the variant sequence is about 85%, 88%, 90%, 95%, 99% or more homologous to SEQ ID NO. 1 to SEQ ID NO. 14. In an embodiment, the variant sequence is about 85%, 88%, 90%, 95%, 99% or more homologous to SEQ ID NO. 5.

The term “cyclized peptide” or similar refers to peptides in which a bridge or a link is formed between two amino acids that are part of the peptide or constitute the peptide. The bridge can be formed between amino acids having a reactive group (other than the amino and the carboxyl group that are essential for the respective amino acid), preferably, such as a sulphide group. Generally, peptides comprising two or more, preferably two amino acids having such a reactive group can be cyclized. For example, a peptide comprising two amino acids that have a sulphide group can be cyclized under conditions wherein a disulphide bridge between the sulphide groups of the two amino acids containing a sulphide group is formed. Examples of amino acids having a sulphide group and thus being capable of forming a bridge, i.e. a disulphite bridge include, but are not limited to penicillamine and cysteine. Peptides in which the bonds forming the ring are not solely peptide linkages (or cupeptide linkages according to the IUPAC) are preferably referred to as heterodetic cyclic peptides. In this case, the bonds between the reactive groups (other than the amino and the carboxyl group that are essential for the respective amino acid) forming the ring are preferably referred to as “bridge”. Alternatively, peptides in which the bonds forming the ring are solely peptide linkages (or cupeptide linkages according to the IUPAC) are preferably referred to as homodetic cyclic peptides.

Term, “isosteres” as used herein, modifications that replace the traditional peptide bond with something that isn't sensitive to proteases. Such Peptide-bond isosteres could be for example: thioamides, esters, alkenes, and fluoroalkenes (as detailed in Chembiochem. 2011 Aug. 16;12 (12): 1801-7. doi: 10.1002/cbic.201100272. Epub 2011 Jul. 12. PMID: 21751326; PMCID: PMC3253576), amide bioisostere (as detailed in J. Med. Chem. 2020, 63, 21, 12290-12358).

Term “amino acid” refers to naturally occurring and non-naturally occurring amino acids, as well as imino acids such as proline, amino acid analogs and amino acid mimetics that function in a manner similar to the naturally occurring amino acids.

Naturally encoded amino acids are proteinogenic amino acids known to those of skill in the art. They include the 20 common amino acids (Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.) and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids and acylated amino acids.

The amino acid residues described herein are in the “L” isomeric form unless otherwise indicated, however, residues in the “D” isomeric form can be substituted for any L-amino acid residue, as long as the desired function is retained by the peptide.

Term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof. For example: non-natural amino acids are described in U.S. Pat. No. 9,637,441B2, US20200299403A1, incorporated by reference in their entirety. See also for example, S. Hunt, The Non-Protein Amino Acids: In Chemistry and Biochemistry of the Amino Acids, edited by G. C. Barrett, Chapman and Hall, 1985. Some examples of unnatural amino acids are 4-hydroxyproline, desmosine, gamma-aminobutyric acid, beta-cyanoalanine, norvaline, 4-(E)-butenyl-4 (R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanccarboxylic acid, 4-amino-cyclopentenccarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O) C₆H₅; —CF₃; —CN; -halo; —NO₂; CH₃), disubstituted phenylalanines, substituted tyrosines (e.g., further substituted with —C(═O) C₆H₅; —CF₃; —CN; -halo; —NO₂; CH₃), and statine, N-methyl arginine, K(N₃) as azidolysine. Additionally, the amino acids suitable for use in the present invention may be derivatized to include amino acid residues that are hydroxylated, phosphorylated, sulfonated, acylated, and glycosylated, to name a few. In an embodiment, non-natural amino acids may be D-amino acids.

Term “olefin-terminated amino acid” is a subset of non-naturally amino acids. It refers to olefin metathesis-based strategies involved for modification of amino acids. For example: α,α-disubstituted non-natural amino acids containing alkenyl side chains, (Schafmeister et al., J. Am. Chem. Soc. 2000, 122, 5891; Blackwell et al., Angew. Chem. Intl. Ed. 1994, 37, 3281). Olefin terminated amino acids may be used to form stapled peptides, which in turn may have increase vivo half-life, increase affinity for a receptor by as much as 103-104-fold, etc. In an embodiment, various examples of olefin terminated amino acids may be as disclosed in U.S. Pat. No. 9,458,189B2, incorporated by reference in its entirety.

Term “conservative substitution” refers to a substitution of one residue with a chemically or biologically similar residue. Examples of conservative substitutions include the replacement of a hydrophobic residue, such as isoleucine, valine, leucine, or methionine for another, the replacement of a polar residue for another, such as the substitution of arginine for lysine, glutamic for aspartic acids, or glutamine for asparagine, and the like. Those of skill in the art will recognize the numerous amino acids that can be modified or substituted with other chemically similar residues without substantially altering activity.

Term, “PEG”, “polyethylene glycol” and “poly(ethylene glycol)” as used herein, are meant to encompass any water-soluble poly(ethylene oxide). Typically, PEGs comprise the following structure “—O(CH2CH2O)m-” where (m) is 2 to 4000. As used herein, PEG also includes “—(CH2CH2O)m-” and “—CH2CH2—O(CH2CH2O)m-CH2CH2-”, depending upon whether or not the terminal oxygens have been displaced. The term “PEG” includes structures having various terminal or “end capping” groups and so forth. The term “PEG” also means a polymer that contains a majority, that is to say, greater than 50%, of —CH2CH2O— monomeric subunits. With respect to specific forms, the PEG can take any number of a variety of molecular weights, as well as structures or geometries such as “branched,” “linear,” “forked,” “multifunctional,” “dendrimeric”, and the like.

PEG conjugates are an established method for peptide and protein delivery pioneered by basic research by Davis and Abuchowski (Abuchowski et al., 1977a and 1977b). Various PEG protein conjugates are known to be protected from proteolysis and/or poorly immunogenic (Monfardini et al., 1995; and Yamasuki et al., 1988). U.S. Pat. No. 5,932,462A, U.S. Pat. No. 20050089515, US20040162388A1 and US20040249576A1 related to PEG-peptide conjugates are incorporated by the reference in their entirety. In an embodiment, scaffold doesn't have to PEGylated.

Term, “chemical linker” or “linker” or “linker moiety,” as used herein, refers broadly to a chemical structure that is capable of linking or joining together two peptide monomer subunits to form a dimer. Linker molecules (“linkers”) may be peptide or non-peptide molecule. Examples of peptide linker molecules include glycine-rich peptide linkers (see, e.g., U.S. Pat. No. 5,908,626), wherein more than half of the amino acid residues are glycine.

Linker molecules may also include non-peptide or partial peptide molecules. For instance, the peptide may be linked to other molecules using well known cross-linking molecules such as glutaraldehyde or EDC (Pierce, Rockford, Ill.), azide containing linkers etc. Bifunctional cross-linking molecules are linker molecules that possess two distinct reactive sites. For example, one of the reactive sites of a bifunctional linker molecule may be reacted with a functional group on a peptide to form a covalent linkage and the other reactive site may be reacted with a functional group on another molecule to form a covalent linkage. General methods for cross-linking molecules have been reviewed (see, e.g., Means and Feeney, Bioconjugate Chem., 1:2-12 (1990)). Homobifunctional cross-linker molecules have two reactive sites which are chemically the same. Examples of homobifunctional cross-linker molecules include, without limitation, glutaraldehyde; N,N′-bis(3-malcimido-propionyl-2-hydroxy-1,3-propanediol (a sulfhydryl-specific homobifunctional cross-linker); certain N-succinimide esters (e.g., discuccinimyidyl suberate, dithiobis(succinimidyl propionate), and soluble bis-sulfonic acid and salt thereof (see, e.g., Pierce Chemicals, Rockford, Ill.; Sigma-Aldrich Corp., St. Louis, Mo.). A bifunctional cross-linker molecule is a heterobifunctional linker molecule, meaning that the linker has at least two different reactive sites, each of which can be separately linked to a peptide or other molecule. Use of such heterobifunctional linkers permits chemically separate and stepwise addition (vectorial conjunction) of each of the reactive sites to a selected peptide sequence. Heterobifunctional linker molecules include, without limitation, m-maleimidobenzoyl-N-hydroxysuccinimide ester (see, Green et al., Cell, 28:477-487 (1982); Palker et al., Proc. Natl. Acad. Sci (USA), 84:2479-2483 (1987)); m-maleimido-benzoylsulfosuccinimide ester; malcimidobutyric acid N-hydroxysuccinimide ester; and N-succinimidyl 3-(2-pyridyl-dithio) propionate (see, e.g., Carlos et al., Biochem. J., 173:723-737 (1978); Sigma-Aldrich Corp., St. Louis, Mo.).

Term, “analog” as used herein refers to analog of an amino acid, e.g., a “Phe analog” or a “Tyr analog” means an analog of the referenced amino acid. A variety of amino acid analogs are known and available in the art, including Phe and Tyr analogs. In certain embodiments, an amino acid analog, e.g., a Phe analog or a Tyr analog comprises one, two, three, four or five substitutions as compared to Phe or Tyr, respectively. In certain embodiments, the substitutions are present in the side chains of the amino acids.

Term, “modified nucleic acid polymer” or similar term as used herein refers to a modified nucleic acid polymer refers to a nucleic acid chain, such as DNA or RNA, that has been chemically altered or modified from its natural form. These modifications can be introduced to confer specific properties or functionalities to the nucleic acid, such as but not limited to base modifications; sugar modifications such as 2′-O-methyl, 2′-fluoro, and 2′-amino substitutions in RNA; phosphate modifications such as phosphorothioate linkages; Locked Nucleic Acids (LNAs); fluorescent or Conjugated Modifications etc.

In an embodiment, the chemical modification may involve copper-free click chemistry as described in U.S. Pat. No. 8,808,665B2. In an embodiment, analogs described in U.S. Pat. No. 10,117,840B2 are incorporated by reference in its entirety.

An embodiment of the disclosure relates to an agonist of PD-1 molecules that would stimulate the effects of natural PD-1/PD-L1 binding, leading to enhanced immune suppression.

In an embodiment of the disclosure, we endeavoured to develop a peptide-based PD-1 agonist, based on the natural sequence of PD-L1 (SEQ ID NO. 1).

Till date, no direct peptide-based PD-1 agonists have been described in literature. Peptides of the present invention have the distinct advantage of being simpler and easier to synthesize than proteins or antibodies, thus reducing the costs of an eventual therapeutic.

### Peptide

In an embodiment, a peptide disclosed herein, or the variant thereof could form a complex with the PD-1.

An embodiment relates to a variant peptide of SEQ ID NO. 1, wherein the variant peptide is at least 85% homologous to SEQ ID NO. 1.

In an embodiment, the variant peptide is configured to bind with molecules of programmed death protein-1 (PD-1) to form a complex that reduces release of an inflammatory cytokine from a cell.

In an embodiment, a peptide mentioned in this disclosure is a PD-1 agonist.

In an embodiment, a peptide mentioned herein, or the variant thereof could be synthesized as per understanding of a person skilled in the art.

In an embodiment, one or more peptides mentioned herein, or the variant thereof are either cyclized or linearized.

In an embodiment, one or more peptides mentioned herein, or the variant thereof can be cyclized in different ways:
(a) Thiol oxidation (disulfide bridge formation) of two cysteines (C) contained in the peptide,
For example, thiol oxidation via a C at or near the N-terminus (of the peptide sequence before cyclization) and a C at or near the C-terminus;
(b) amide condensation of two amino acid side chains (lactam),
For example, amide condensation via lysine (K) and aspartate (D) side chains,
For example, amide condensation via the K side chain at or near the N-terminus (of the peptide sequence before cyclization) and the D side chain at or near the C-terminus;
(c) head-to-tail cyclization;
For example, head-to-tail cyclization via the N-terminus (of the peptide sequence before cyclization), i.e. the amino group of the N-terminal amino acid and the C-terminus, i.e. the carboxyl group of the C-terminal amino acid;
For example, head-to-tail cyclization via lysine (K) at the N-terminus and C-terminus (of the peptide sequence before cyclization);
(d) skeleton cyclization;
(e) thioether formation;
and/or (f) cyclization by hydrogen bond forming and/or bond forming derivatives of amino acids, eg. forming tryptophan zippers.

In an embodiment, one or more residues may be added in one or more peptides mentioned herein, or the variant thereof to allow desired cyclization. For example: the disulphide bond or the thioether bond is formed via addition of a residue in the peptide or the variant thereof. For example: In SEQ ID NO. 9, (NH3-ARAMISYGGADYK-(N-Me-Arg)-IC—COOH), where a thioether bond is established between the C-terminal cysteine residue and the N-terminal A residue. In another example, NH3-CRAMISYGGADYK-(N-Me-Arg)-IC—COOH (SEQ ID NO. 5), in which the N-terminal and C-terminal residues form a disulfide bond to cyclize the peptide.

In an embodiment, mutation of one or more residues in a peptide mentioned herein, or the variant thereof is also allowed. Such as but not limited to mutation having one or more substitutions, deletions and/or additions relative to SEQ ID NO. 1 that retain the ability to bind PD-1 and trigger a signal through PD-1.

In an embodiment, one or more residue of the peptide mentioned herein, or the variant thereof may be chemically modified or include chemical derivatives, in which one or more of the amino acids therein have a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxyl groups that form O-acyl or O-alkyl derivatives, as well as naturally occurring amino acid derivatives, for example, 4-hydroxyproline, for proline, 5-hydroxylysine for lysine, homoserine for serine, ornithine for lysine, and so forth. Also included are D-amino acids and amino acid derivatives that can alter covalent bonding, for example, the disulfide linkage that forms between two cysteine residues that produce a cyclized polypeptide.

In an embodiment, chemical modification may be conversion of arginine to N-methyl arginine, such as ‘X’ represented in SEQ ID NO. 5

In an embodiment, such non-peptide modification may help to maintain or improve serum stability of the peptide of the invention. Chemical modification may help the peptide from degradation.

Chemical modification according to present invention includes but not limited to:

Phosphorylation: Addition of a phosphate group to specific amino acids, such as serine, threonine, and tyrosine. Phosphorylation plays a crucial role in signalling pathways, protein regulation, and cellular processes like cell division and metabolism.

Glycosylation: Attachment of sugar molecules to specific amino acids, typically serine, threonine, or asparagine residues. Glycosylation is essential for protein folding, stability, and recognition, as well as cell-cell adhesion and signalling.

Acetylation: Addition of an acetyl group to the amino terminus of a protein or to specific lysine residues. Acetylation can regulate protein stability, localization, and activity, as well as influence chromatin structure and gene expression.

Methylation: Addition of methyl groups to specific amino acids, such as lysine and arginine. Methylation can regulate protein-protein interactions, chromatin structure, and gene expression, among other cellular processes.

Hydroxylation: Addition of hydroxyl groups to certain amino acids, commonly proline and lysine. Hydroxylation is crucial for collagen synthesis, stabilization of protein structure, and regulation of protein-protein interactions.

Ubiquitination: Attachment of ubiquitin molecules to lysine residues, targeting proteins for degradation by the proteasome or regulating their activity, localization, and interactions within cells.

Sulfation: Addition of sulfate groups to tyrosine residues. Sulfation is involved in the regulation of protein-protein interactions, signal transduction, and extracellular matrix organization.

Carboxylation: Addition of carboxyl groups to glutamate residues. Carboxylation is essential for the activity of certain proteins involved in blood clotting and bone formation.

In an embodiment, arginine in SEQ ID NO. 1 is substituted with N-methyl arginine. In an embodiment, arginine towards C-terminal in SEQ ID NO. 1 is substituted with N-methyl arginine. Arg125 of SEQ ID NO. 1 is substituted with N-methyl Arg125. Methylation of amino acids protects the peptide from degradation.

In an embodiment, a peptide mentioned herein, or the variant thereof can be altered by deleting one or more residues from N- and/or C-terminal ends. For example: one or two residues deleted from N- or C-terminal ends of the peptide. In another example, one residue from N-terminal and another deletion of residue from C-terminal from the peptide is also possible. In another case, two or more residues are deleted from N-terminal end. In another case, two or more residues are deleted from C-terminal residues.

In an embodiment, peptide could be —NH3-CRAMISYGGADYK-(N-Me-Arg)-IC—COOH, in which the N-terminal and C-terminal residues form a disulfide bond to cyclize the peptide. (SEQ ID NO. 5).

In an embodiment, the peptide is cyclized through the addition of residues for disulfide bonds or thioether bonds.

In an example of the above embodiment, the sequences could be NH3-ARAMISYGGADYK-(N-Me-Arg)-IC—COOH (SEQ ID NO. 9), where a thioether bond is established between the C-terminal cysteine residue and the N-terminal A residue.

In an embodiment, the peptide could be truncated such that multiple amino acids from either the N-terminus or C-terminus are removed, or the N-terminal and C-terminal amino acids are protected via acetylation or amidation.

In an embodiment, the peptides are modified to include azido-lysine residues to accommodate attachment to a scaffold. The Scaffold may include but not limited to a modified semi rigid nucleic acid polymer, such as PEGylated DNA or RNA. In another embodiment, scaffolding unit comprised of a modified semi-rigid nucleic acid backbone such as peptide-nucleic acid (PNA) or phosphorothioate-polymerized nucleotides rather than phosphodiester-polymerized nucleotides.

In an embodiment, the N-terminal and C-terminal amino acids of the peptide mentioned herein, or the variant thereof are protected via acetylation or amidation. For example: the sequences could be Acetyl-CRAMISYGGADYK-(N-Me-Arg)-IC-Amide (SEQ ID NO. 10), in which the N-terminal and C-terminal residues for a disulfide bond to cyclize the peptide.

In an embodiment, one or more amino acids of the peptide may have a conservative substitution.

In an embodiment, a peptide mentioned herein, or the variant thereof may be modified by a non-peptide bond modification, such as D-enantiomers, N-methylated groups, PEGylation (attachment of PEG molecules to amino acids), isosteres or a combination thereof.

In an embodiment, a peptide mentioned herein thereof may be modified by attaching to the peptide a scaffolding unit via chemical linkers including, but not limited to, azido groups, and copper-free click-chemistry reactions. For example: a sequence is modified to include azido-lysine [K(N3)] residues to accommodate attachment to a scaffold. For example: X1 of SEQ ID NO. 11 is K(N3) azido-lysine residue as shown having NH3-K(N3)-CRAMISYGGADYK-(N-Me-Arg)-IC—COOH (SEQ ID NO. 11). In SEQ ID NO. 11, a disulfide bond is established between the C-terminal cysteine residue and the N-terminal residue.

In an embodiment, a peptide mentioned herein, or the variant thereof may be modified by including analogs. These analogs could provide additional metabolic stability. Some examples of analogs are: D-amino acid analog, peptidomimetics, peptide conjugates, fluorinated peptides, peptide hybrids etc. In an embodiment, a peptide analog includes fluorine.

In another embodiment, a peptide mentioned herein, or the variant thereof are modified to incorporate any number of additional amino acids or replacement of amino acids to allow for staples or branches to increase rigidity of the peptide.

In an example of the above embodiment, the sequences could be NH3-K(N3)-CRA*-MISYGGADYK-(N-Me-Arg)-I*C—COOH (SEQ ID NO. 12) in which A* and I* are olefin-terminated, non-natural amino acids in place of the original A and I to allow for staples prepared by ring closing metathesis.

In an example of the above embodiment, the sequences could be NH3-K(N3)-CRAMIS*YGGA*DYK-(N-Mc-Arg)-IC—COOH (SEQ ID NO. 13) in which S* and A* are olefin-terminated, non-natural amino acids in place of the original S and A to allow for staples prepared by ring closing metathesis.

In an embodiment, at least one of peptides or variant thereof selected from the SEQ ID NO. 1 to 13 is a PD-1 agonist.

In an embodiment, variant peptide includes peptides selected from SEQ ID NO. 2 to 13. In an embodiment, the peptide of SEQ ID NO. 5 is a PD-1 agonist.

In an embodiment, X14 in SEQ ID NO. 6 is norleucine (Nlc).

In an embodiment, mutation of some residues in SEQ ID NO. 1 may not be permitted. For example: in one aspect, mutation at residue at 2nd position is not allowed. In another aspect, mutation at residue 4 is not allowed. Yet in another aspect, mutation at mutation at residue 11 is not allowed. Yet in another aspect, mutation at mutation at residue 12 is not allowed. Yet in another aspect, mutation at mutation at residue 13 is not allowed. Yet in another aspect, mutation at mutation at residue 14 is not allowed.

In an embodiment, mutation at Y12A in SEQ ID NO. 1 is not allowed.

In an embodiment, mutation at R12A in SEQ ID NO. 1 is not allowed.

In an embodiment, Valine residue in SEQ ID NO. 1 is removed.

TABLE 1
List of peptide sequences

| SEQ | | N- | C- | Our | Definition of |
|---|---|---|---|---|---|
| ID NO. | Sequence | term | term | Nomenclature | ‘X’ residue |
| 1 | YRCMISYGGADYKRITV | NH₂ | COOH | | — |
| 2 | YRCMISYGGADYKRITV | acetyl | amide | MN1.1 | — |
| 3 | YRCMISNGGADYKRITV | acetyl | amide | MN 2.1 | — |
| 4 | CYRAMISYGGADYKRITVC | Disulfide | | MN 1.2 | — |
| | | cyclization | | | |
| | | Head to Tail | | | |
| 5 | CRAMISYGGADYKXIC | Disulfide | | MN1.4 | X14-N-Me- |
| | | cyclization | | | Arg- |
| | | Head to Tail | | | |
| 6 | CRAXISYGGADYKXIC | Disulfide | | MN1.5 | X14-N-Me- |
| | | cyclization | | | Arg; |
| | | Head to Tail | | | X4: Nor- |
| | | | | | leucine |
| 7 | CRAMISYKGADYKRIC | Disulfide | | MN 3.2 | — |
| | | cyclization | | | |
| | | Head to Tail | | | |
| 8 | CRALIAYKGADYKRIC | Disulfide | | MN4.2 | — |
| | | cyclization | | | |
| | | Head to Tail | | | |
| 9. | ARAMISYGGADYKXIC | NH₂ | COOH | | X- N-Me-Arg; |
| 10. | CRAMISYGGADYKXIC | Disulfide | | | X14- N-Me- |
| | | cyclization | | | Arg; |
| | | Head to Tail | | | |
| 11. | XCRAMISYGGADYKXIC | Disulfide | | | X1- K(N₃)- |
| | | cyclization | | | X15- N-Me- |
| | | | | | Arg; |
| 12 | XCRXMISYGGADYKXXC | acetyl | amide | | X1- K(N₃)- |
| | | | | | X4-olefin- |
| | | | | | terminated, |
| | | | | | non-natural |
| | | | | | amino acids |
| | | | | | X15-N-Me- |
| | | | | | Arg; |
| | | | | | X16-olefin- |
| | | | | | terminated, |
| | | | | | non-natural |
| | | | | | amino acids |
| 13. | XCRAMIXYGGXDYKXIC | acetyl | amide | | X1-K(N3)- |
| | | | | | X7-olefin- |
| | | | | | terminated, |
| | | | | | non-natural |
| | | | | | amino acids; |
| | | | | | X11-olefin- |
| | | | | | terminated, |
| | | | | | non-natural |
| | | | | | amino acids |
| | | | | | X15-N-Me- |
| | | | | | Arg; |
| 14. | YRCMISYGGADEKRITV | acetyl | amide | MN_null | |

### Properties of Peptide

In an embodiment, a peptide mentioned herein, or the variant thereof binds with molecules of programmed death protein-1 (PD-1) to form a complex that modulate release of an inflammatory cytokine from a cell. In an embodiment, such cells could be cancerous cells. Programmed death protein-1 (PD-1) may be in its native 3-D configuration.

In an embodiment, the complex leads to reduction of an inflammatory cytokine from a cell in a dose dependent manner. That means, reduction of the inflammatory cytokine increases with the increase in dosage of the peptide.

In an embodiment, activity of decrease of inflammatory cytokine by a peptide mentioned herein, or the variant thereof is comparable to a recombinant PD-L1. That means a peptide of the disclosure may be decreasing the same inflammatory cytokine as being decreased by the recombinant PD-L1, for example: both recombinant PD-L1 and the peptide of the invention decreases TNFα in a cancer cell line. In another embodiment, the peptide of the disclosure may act differently compared to that of recombinant PD-L1. For example: peptide of the present invention may be decreasing a different inflammatory cytokine compared to the recombinant PD-L1 in a cell line. For example: the peptide may decrease IL-2, whereas the recombinant PD-L1 may decrease TNFα in a cancer cell line.

In an embodiment, the peptide of the disclosure or the variant thereof is more potent compared to the recombinant PD-L1 in decreasing the inflammatory cytokine. That means, a lesser concentration of the peptide of the disclosure of variant thereof provides same or similar result provided by recombinant PD-L1.

In an embodiment, a variant peptide comprising SEQ ID NO. 5 is equal or more potent compared to SEQ ID NO. 1, That means, a lesser concentration of the peptide of the disclosure of variant thereof provides same or similar result provided by SEQ ID NO. 1. A concentration of the variant peptide comprising SEQ ID NO. 5 could be 1 μM, 5 μM, 10 μM, 20 μM, 25 μM giving same or similar result as that by SEQ ID NO. 1.

In an embodiment, a variant peptide comprising SEQ ID NO. 5 is equal or more potent compared to SEQ ID NO. 2, That means, a lesser concentration of the peptide of the disclosure of variant thereof provides same or similar result provided by SEQ ID NO. 1. A concentration of the variant peptide comprising SEQ ID NO. 5 could be 1 μM, 5 μM, 10 μM, 20 μM, 25 μM giving same or similar result as that by SEQ ID NO. 2.

In an embodiment, about 1 μM, 5 μM, 10 μM, 20 μM, 50 μM, 100 μM, 500 μM, 1000 μM or more of the peptide of the disclosure or the variant thereof could decrease release of an inflammatory cytokine. In an embodiment, the decrease in the inflammatory cytokine could be about 10%, 20%, 30%, 40%, 50%, 60%, 70%, 80%, 90%, 95% or more in a cell treated with the peptide or the variant thereof disclosed in the disclosure.

In an embodiment, the peptide of the disclosure of variant thereof is stable in a serum. Serum could be human serum. The stability of the peptide could be for about 15 mins, 30 mins, 60 mins, 1 hour, 2 hours, 10 hours, 20 hours, 30 hours, 40 hours, 50 hours, 60 hours, 70 hours, 80 hours, 90 hours, 100 hours, 120 hours, 150 hours, 200 hours or more. In an embodiment, the peptide may be stable in human serum for about 1, 2, 4, 8, 10, 20, 30 days or more.

In an embodiment, the peptide of the disclosure of variant thereof degrades less than selected from about 5%, 10%, 15%, 20%, 30%, 40%, 50% in a human serum for a time-period about 15 mins, 30 mins, 60 mins, 1 hour, 2 hours, 10 hours, 20 hours, 30 hours, 40 hours, 50 hours, 60 hours, 70 hours, 80 hours, 90 hours, 100 hours, 120 hours, 150 hours, 200 hours 1, 2, 4, 8, 10, 20, 30 days or more.

In an embodiment, compared to antibody PD-1 agonists, the peptide of the present invention has case of synthesis leading to reduced cost.

In an embodiment, the complex is configured to suppress release of an inflammatory cytokine from a cell.

In an embodiment, the complex is stable in a human body. In another embodiment, the complex is stable in vitro in a vial.

In an embodiment, the variant peptide's binding mode includes noncovalent interactions with lysine 78 molecule on PD-1.

In an embodiment, peptide of the present invention also exhibits in vivo stability, physical and chemical stability, including (but not limited to) thermal stability, solubility, low self-association and can be used for development and/or pharmacokinetic characteristics for the treatment of autoimmune disorders and/or transplant rejection (ie, graft-versus-host disease).

In an embodiment SEQ ID NO. 5 exhibits in vivo stability, physical and chemical stability, including (but not limited to) thermal stability, solubility, low self-association and can be used for development and/or pharmacokinetic characteristics for the treatment of autoimmune disorders and/or transplant rejection (ie, graft-versus-host disease).

In an embodiment, peptide of the present invention has increased simplicity of dosing due to more conventional half-lives versus PD-1 antibodies.

PD-1 antibodies include commercially available antibodies such as but not limited to pembrolizumab and nivolumab.

In some embodiments, the variants of SEQ ID NO. 1 of the present invention exhibit increased stability (e.g., as measured by half-life, rate of protein degradation) as compared to SEQ ID NO. 1. In certain embodiments, the stability of peptide of the present invention is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a reference SEQ ID NO. 1. In some embodiments, the stability is serum stability, e.g., as optionally measured according to the method described herein.

In an embodiment, variants of SEQ ID NO. 1 selected from SEQ ID NO. 2 to SEQ ID NO. 13.

In some embodiments, the variants of SEQ ID NO. 1 of the present invention exhibit increased stability (e.g., as measured by half-life, rate of protein degradation) as compared to PD-1 antibodies. In certain embodiments, the stability of a peptide of the present invention is increased at least about 1.5, 2, 3, 4, 5, 6, 7, 8, 9, 10, 11, 12, 13, 14, 15, 16, 17, 18, 19, 20, 30, 40, 50, 60, 70, 80, 90, 100, 120, 140, 160, 180, or 200-fold greater or at least about 10%, 20%, 30%, 40%, 50%, 60%, 70%, 80%, 90%, 100%, 200%, 300%, 400%, or 500% greater than a reference PD-1 antibodies.

Stability as described above under the given set of conditions wherein the temperature is about 25° C., about 4° C., or about 37° C., and the pH is a physiological pH, or a pH about 7.4.

Advantages of the peptide described herein versus small molecule PD-1 agonists include increased specificity for the target and reduced potential toxicity due to nonspecific binding or toxic metabolites.

### Pharmaceutical Composition

In an embodiment, the present invention provides a composition comprising one or more combinations of the peptides of the present invention or variant thereof as an active ingredient, formulated with a pharmaceutically acceptable carrier, i.e., to provide a pharmaceutical composition.

The pharmaceutical compositions of the present invention can also be administered in combination therapy, for example in combination with other agents. For example, the combination treatment may comprise a peptide of the invention in combination with at least one other anti-inflammatory or immunosuppressive agent. Immunosuppressive agent could be selected according to knowledge of a person skilled in the art.

The term “pharmaceutically acceptable salt” of a given compound refers to salts that retain the biological effectiveness and properties of the given compound, and which are not biologically or otherwise undesirable (see e.g., Berge, S. M., et al. (1977) J. Pharm. Sci. 66:1-19). Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di (substituted alkyl)amines, tri (substituted alkyl)amines, alkenyl amines, dialkenyl amines, trialkenyl amines, substituted alkenyl amines, di (substituted alkenyl)amines, tri (substituted alkenyl)amines, cycloalkyl amines, di(cycloalkyl) amines, tri (cycloalkyl) amines, substituted cycloalkyl amines, di-substituted cycloalkyl amine, tri-substituted cycloalkyl amines, cycloalkenyl amines, di(cycloalkenyl) amines, tri (cycloalkenyl) amines, substituted cycloalkenyl amines, di-substituted cycloalkenyl amine, tri-substituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from alkyl, substituted alkyl, alkenyl, substituted alkenyl, cycloalkyl, substituted cycloalkyl, cycloalkenyl, substituted cycloalkenyl, aryl, heteroaryl, heterocyclic. Also included are amines where the two or three substituents, together with the amino nitrogen, form a heterocyclic or heteroaryl group. Amines are of general structure N(R30) (R31) (R32), wherein monosubstituted amines have two of the three substituents on nitrogen (R30, R31, and R32) as hydrogen, di-substituted amines have one of the three substituents on nitrogen (R30, R31, and R32) as hydrogen, whereas tri-substituted amines have none of the three substituents on nitrogen (R30, R31, and R32) as hydrogen. R30, R31, and R32 are selected from a variety of substituents such as hydrogen, optionally substituted alkyl, aryl, heteroaryl, cycloalkyl, cycloalkenyl, heterocyclyl.

Specific examples of suitable amines include, by way of example only, isopropyl amine, trimethyl amine, diethyl amine, tri (iso-propyl) amine, tri (n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethane sulfonic acid, p-toluene-sulfonic acid, salicylic acid.

Pharmaceutically acceptable carriers include, for example, stabilizers, solubilizers, suspending agents, emulsifiers, soothing agents, buffers, preservatives, preservatives, pH adjusters, antioxidants, and the like. Examples of the stabilizer include various amino acids, albumin, globulin, gelatin, mannitol, glucose, dextran, ethylene glycol, propylene glycol, polyethylene glycol, ascorbic acid, sodium bisulfite, sodium thiosulfate, sodium edetate, sodium citrate, Dibutylhydroxytoluene or the like. Examples of solubilizers include alcohols (eg, ethanol), polyalcohols (eg, propylene glycol, polyethylene glycol, etc.), nonionic surfactants (cg, polysorbate 80 (trade name), HCO-50, etc.) Etc. As the suspending agent, for example, glyceryl monostearate, aluminum monostearate, methyl cellulose, carboxymethyl cellulose, hydroxymethyl cellulose, sodium lauryl sulfate and the like. As the emulsifier, for example, gum arabic, sodium alginate, tragacanth and the like. As the soothing agent, for example, benzyl alcohol, chlorobutanol, sorbitol and the like. Examples of the buffer include phosphate buffer, acetate buffer, borate buffer, carbonate buffer, citrate buffer, Tris buffer, glutamate buffer, epsilon aminocaproate buffer, and the like. Preservatives include, for example, methyl paraoxybenzoate, ethyl paraoxybenzoate, propyl paraoxybenzoate, butyl paraoxybenzoate, chlorobutanol, benzyl alcohol, benzalkonium chloride, sodium dehydroacetate, sodium edetate, boric acid, boron Sand or the like. As the preservative, for example, benzalkonium chloride, paraoxybenzoic acid, chlorobutanol and the like. As the pH adjuster, for example, hydrochloric acid, sodium hydroxide, phosphoric acid, acetic acid and the like. Antioxidants include, for example, (1) water-soluble antioxidants such as ascorbic acid, cysteine hydrochloride, sodium bisulfate, sodium metabisulfite, sodium sulfite, etc., (2) ascorbyl palmitate, butylated hydroxyanisole, Use oil-soluble antioxidants such as butylated hydroxytoluene, lecithin, propyl gallate, α-tocopherol, and (3) metal chelating agents such as citric acid, ethylenediaminetetraacetic acid, sorbitol, tartaric acid, phosphoric acid, etc.

In an embodiment, therapeutic amount could be as described in U.S. Pat. No. 9,701,749B2, U.S. Pat. No. 9,580,507B2, US20110229461A1, etc. which are incorporated by reference in its entirety.

The composition may also be formulated as a solution, microemulsion, liposome or other structure suitable for high drug concentration. The carrier may be, for example, a solvent or dispersion medium comprising water, ethanol, polyol (eg, glycerol, propylene glycol, liquid polyethylene glycol, etc.), nanoparticles and suitable mixtures thereof. Proper flowability can be maintained, for example by using a coating material such as lecithin, by maintaining the required particle size in the case of dispersion, and by the use of surfactants. In many cases, it will be desirable to include in the composition, for example, polyalcohols such as sugar, mannitol, sorbitol, or isoelectric agents such as sodium chloride. Delayed absorption of the injectable composition can occur by including in the composition an agent that delays absorption, for example with a monostearate salt and gelatin.

The amount of active ingredient that can be combined with a carrier material to produce a single dosage form will vary depending on the subject being treated, in particular the form of administration. The amount of active ingredient that can be combined with a carrier material to produce a single dosage form will generally be the amount of the composition that produces a therapeutic effect. Generally, out of 100 percent, this amount is about 0.01 percent to 99 percent active ingredient, preferably about 0.1 percent to 70 percent, and most preferably about 1 percent to 30 percent active ingredient, which is pharmaceutically acceptable. It will be a range in combination with a carrier.

The actual dosage level of the active ingredient in the pharmaceutical composition of the present invention can vary in various ways to obtain the amount of active ingredient effective to achieve the desired therapeutic response to a particular patient, composition and mode of administration without being toxic to the patient. The dosage level selected is used in combination with the specific composition of the invention used, the activity of its esters, salts or amides, the route of administration, the time of administration, the excretion rate of the specific compound used, the duration of treatment, and the specific composition used. Other drugs, compounds and/or substances that are treated, the age, sex, weight, condition, general health and previous medical history of the patient being treated, and a variety of pharmacokinetic factors, including other factors well known in the medical field, will be.

By a “therapeutically effective amount” or similar of a composition of the invention is meant an amount of the composition which confers a therapeutic effect on the treated subject, at a reasonable benefit/risk ratio applicable to any medical treatment. The therapeutic effect is sufficient to “treat” the patient as that term is used herein. For example, the “therapeutically effective dose” is at least about 20%, preferably at least about 40%, more preferably at least about 60%, even more preferably at least about 80% or more, suppresses release of one or more inflammatory cytokine compared to an untreated subject. The ability of a compound to decrease cytokine production can be evaluated in a diagnostic animal model system for efficacy in human tumors. Alternatively, the properties of this composition can be assessed by examining the ability of the compound to inhibit, such as inhibition in vitro, by assays known to the skilled practitioner. One of ordinary skill in the art will be able to determine this amount based on factors such as the size of the subject, the severity of the subject's symptoms, and the route of administration selected or the particular composition.

In an embodiment, a therapeutically effective amount could be in the range of about 0.0001-100 mg kg, and more often 0.01-5 mg/kg of the host body weight. For example, doses can be 0.3 mg/kg body weight, 5 mg/kg body weight, or 10 mg/kg body weight, or in the range of 1-10 mg/kg.

An approximate treatment regimen includes administration once a day, once a week, once every two weeks, once every three weeks, once every four weeks, once a month, once every 3 months, or once every 3-6 months. In an embodiment, if the pharmaceutical is orally available, it may be advantageous to dose once a day, at lower conc in an early period of treatment, then taper back as treatment progresses.

The compositions of the present invention may be administered through one or more routes of administration using one or more of a variety of methods known in the art. As will be appreciated by the skilled practitioner, the route and/or mode of administration will vary depending on the desired outcome. Preferred routes of administration of the pharmaceutical composition of the invention include intravenous, intramuscular, intraepithelial, intraperitoneal, subcutaneous, spinal cord or other parenteral routes of administration, for example by injection or infusion. As used herein, the phrase “parenteral administration” refers to a mode of administration other than intravenous and local administration by injection, without limitation, intravenous, intramuscular, intraarterial, intracapsular, intracapsular, Intraorbital, intracardiac, intraepithelial, intraperitoneal, via conduit, subcutaneous, subcuticle, intraarticular, subcapsular, subarachnoid, intrathecal, epidural and intrasternal injections and infusions.

Alternatively, the pharmaceutical composition of the invention may be topically, via non-intestinal routes such as epithelial or mucosal routes of administration, for example nasal, oral, vaginal arteries, rectal, sublingual or topical. It can be administered as.

The active ingredients can be prepared with carriers that protect compounds such as implants, patches through the skin, and microencapsulated delivery systems against rapid release. Biodegradable and biocompatible polymers such as ethylene vinyl acetate, polyanhydrides, polyglycolic acid, collagen, polyorthoester, and polylactic acid may be used. Many methods for the preparation of such formulations are patented or are generally known to those skilled in the art. See, for example, Sustained and Controlled Release Drug Delivery Systems, J. R. See Robinson, ed., Marcel Dekker, Inc., New York, 1978.

In an embodiment, the composition is therapeutically more effective than rPD-L1.

### Method of Treatment

The peptides of the present invention selected from SEQ ID NO. 1 to 13 or variant thereof and methods described have numerous in vitro and in vivo utilities involving, for example, suppression of immune response by binding to PD-1. In an embodiment, the peptide of SEQ ID NO. 5 attached to PD-1 to suppress immune response. In an embodiment, these peptides can be administered to cells in culture, in vitro or ex vivo, or to human subjects, e.g., in vivo, to suppress immunity in a variety of situations. Accordingly, in one aspect, the invention provides a method of modifying an immune response in a subject comprising administering to the subject the peptide selected from SEQ ID NO. 1 to 13 or variant thereof of the invention such that the immune response in the subject is modified. Preferably, the response is suppressed or inhibited. In an embodiment, a pharmaceutical composition comprising one or more peptides as an active ingredient working as a PD-1 agonist.

As used herein, the term “subject” is intended to include human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of suppression of an immune response.

In an embodiment, the peptide of the present invention or the variant thereof may be used to treat immune related disease.

The terms “treatment” “treat” and “treating” encompasses alleviation, cure or prevention of at least one symptom or other aspect of a disorder, disease, illness or other condition (collectively referred to herein as a “condition”), or reduction of severity of the condition, and the like. A composition of the invention need not affect a complete cure, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. Beneficial or desired clinical results include, but are not limited to, alleviation of symptoms, diminishment of extent of disease, stabilization (i.e., not worsening) of disease, delay or slowing of disease progression, amelioration or palliation of the disease state, remission (whether partial or total, whether detectable or undetectable) and prevention of relapse or recurrence of disease. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient.

“Treatment” can also mean prolonging survival as compared to expected survival if not receiving treatment. In one embodiment, an indication that a therapeutically effective amount of a composition has been administered to the patient is a sustained improvement over baseline of an indicator that reflects the severity of the particular disorder.

The term “Immune-related disease” means a disease in which the immune system is involved in the pathogenesis of the disease. Subsets of immune-related diseases are autoimmune diseases. Autoimmune diseases include, but are not limited to, rheumatoid arthritis, myasthenia gravis, multiple sclerosis, psoriasis, systemic lupus erythematosus, autoimmune thyroiditis (Hashimoto's thyroiditis), Graves' disease, inflammatory bowel disease, autoimmune uveoretinitis, polymyositis, and certain types of diabetes. Other immune-related diseases are provided infra. In view of the present disclosure, one skilled in the art can readily perceive other autoimmune diseases treatable by the compositions and methods of the present invention. Disease and disorder is interchangeably used through out the specification.

Examples of autoimmune diseases that can be prevented, suppressed and/or treated by the PD-1 agonist in the present invention include Behcet's disease, systemic lupus erythematosus, multiple sclerosis (systemic sclerosis, progressive systemic sclerosis). Disease), scleroderma, polymyositis, dermatomyositis, nodular periarteritis (nodular polyarteritis, microscopic polyangiitis), aortitis syndrome (Takanian arteritis), malignant rheumatoid arthritis, rheumatoid arthritis, Wegener Granulomatosis, mixed connective tissue disease, Sjogren's syndrome, adult Still's disease, allergic granulomatous vasculitis, hypersensitivity vasculitis, Kogan syndrome, RS3PE, temporal arteritis, polymyalgia rheumatica, fibromyalgia Discase, antiphospholipid antibody syndrome, cosinophilic fasciitis, IgG4-related diseases (eg primary sclerosing cholangitis, autoimmune pancreatitis, etc.), Guillain-Barre syndrome, severe Myasthenia, chronic atrophic gastritis, autoimmune hepatitis, primary biliary cirrhosis, aortitis syndrome, Goodpasture syndrome, rapidly progressive glomerulonephritis, megaloblastic anemia, autoimmune hemolytic anemia, self Immune neutropenia, idiopathic thrombocytopenia purpura, Graves' disease (hyperthyroidism), Hashimoto disease, autoimmune adrenal insufficiency, primary hypothyroidism, idiopathic Addison's disease (chronic adrenal cortex) Hypofunction), type I diabetes, chronic discoid lupus erythematosus, localized scleroderma, psoriasis, psoriatic arthritis, pemphigus, pemphigoid, gestational herpes zoster, linear IgA bullous dermatosis, acquired epidermis blisters Disease, alopecia arcata, vulgaris vulgaris, Harada disease, autoimmune optic neuropathy, idiopathic azoospermia, habitual abortion, inflammatory bowel disease (ulcerative colitis, Crohn's disease) and the like. The formulation of the present invention can also be applied to the prevention or treatment of graft-versus-host disease (GVHD).

In an embodiment, peptide of the present invention such as SEQ ID NO. 5 could be as a therapeutic for a number of different disease areas, including but not limited to diabetes, arthritis, lupus, and chronic pain. Further development including additional mutations for affinity or stability, as well as validation in an appropriate animal model for some of the diseases above, could be used to prepare a compelling pre-clinical package.

The invention is described in more detail by the following examples which are not to be construed as additional limitations. All drawings and all references, patents and published patent applications cited throughout this application specification are expressly incorporated herein by reference.

### Example 1: Optimization via Molecular Dynamics Simulations and Computational Alanine Scanning

Interaction interface between PD-1 and PD-L1 was characterized using protein painting, a novel structural biology technique developed in-house (21,34). Protein painting utilizes small molecular dyes that coat solvent-accessible regions of protein complexes and block trypsin digestion in these areas. Only regions of protein-protein interaction, which are solvent-inaccessible and therefore uncoated by dye, are detected by mass spectrometry and are reported as hotspots of interaction.

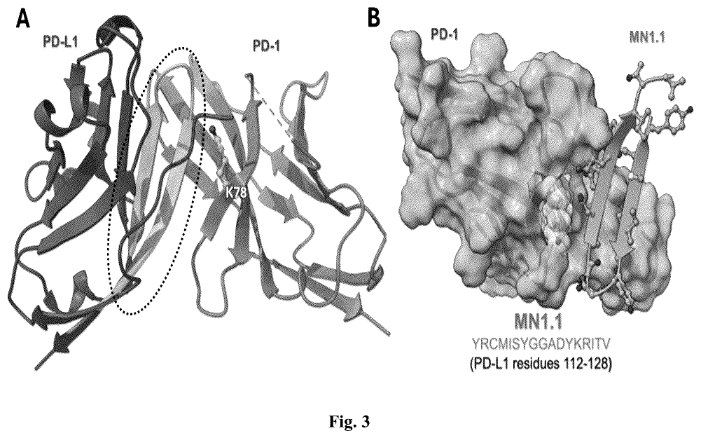

Lys78 on PD-I was identified as a hotspot of interaction, and eight first-generation peptides were designed based on the sequences of the interface regions identified for PD-1 and PD-L121. The most promising first-generation peptide, MN1.1, was designed based on the PD-L1 interface region (residues 112-128). This finding was further supported by the published crystal structure of human PD-1/PD-L1, which showed that the region of PD-L1 used to design interacted directly with Lys78 in the PD-1 interface region (PDB: 4ZQK18). The region of PD-L1 used to design MN1.1 is highlighted in the structure of the PD-1/PD-L1 complex shown in FIG. 3.

A peptide derived directly from the native sequence of PD-L1, predicted to interact with PD-1/PD-L1 binding hotspot Lys78, can inhibit binding of native human PD-1 and PD-L1 (15). Using MN1.1 molecule as the starting point, optimization was subsequently conducted to stabilize the molecule for more complex in-vitro efficacy testing.

The most effective peptide was MN 1.1 head-to-tail disulfide bond cyclized version, MN1.2, designed based on the PD-L1 interface region that interacts directly with K78 on PD-1, with IC50 values under 10 μM.

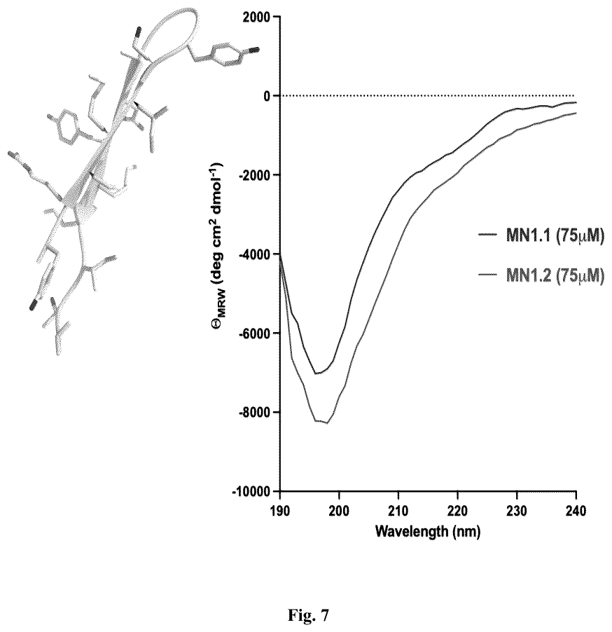

Our collaborators at Virginia Tech performed molecular dynamics simulations to predict the structure of MN1.1 in solution. The modelled structure was then validated via circular dichroism spectroscopy and compared with the head-to-tail disulfide bond cyclized version, MN1.2, as shown in FIG. 7.

Results of molecular dynamics simulations showed MN1.1 adopts mostly a random coil structure in solution. This modelled structure was consistent with the results of CD experiments.

In the ˜220-210 nm range, random coils have a characteristic, shallow positive peak across this range while β-sheets and have a negative peak in this range. Between ˜200-195 nm, where β-sheets have a positive peak while random coils show a negative peak in this range. The CD data demonstrates both MN1.1 and MN1.2 are mostly random coil, with some β-sheet character potentially reducing intensity of the random coil peak between ˜220-230 nm. These results showed that despite the head-to-tail disulfide bond cyclization of MN1.2, the peptide mostly adopted a random coil structure in solution.

Regions of high affinity were not targeted for optimization, given that changes in these regions would be detrimental to peptide affinity for PD-1. A peptide with a predicted null mutation in a high affinity region was generated to validate this data. Regions of low affinity were selected for further optimization, and potential amino acid mutations in selected residues were modelled for affinity.

We conducted molecular dynamics simulations to prepare the lowest energy docked conformations of MN 1.1 with the PD-1 receptor. Subsequently, the per-residue contribution to binding energy was determined, and computational alanine scanning was used to determine a model “null peptide” to validate the modelling system. Positive changes in the affinity of a residue after mutation to alanine indicate the substitution is unfavourable for binding free energy, and therefore suggest the residue is important for binding affinity.

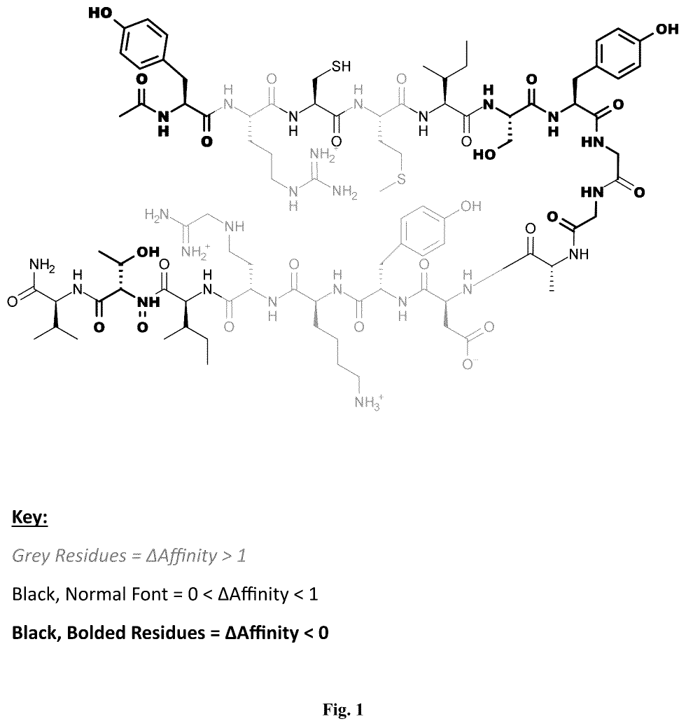

As shown in Table 2 and FIG. 1, computation demonstrated deleterious effects of mutating most residues in the peptide, in particular tyrosine residue 12. This data was validated using an AlphaScreen binding model, which demonstrated agreement with the modelled system. These regions are shown in light grey in FIG. 1. In contrast, there were residues with improved affinity after mutation to alanine, shown in bold black. These residues were thought to be amenable to amino acid substitution for improving the overall affinity of the peptide for PD-1. Further molecular dynamics simulations were performed by our collaborators to choose the specific amino acids substitutions that would be tested.

As shown in Table 2, the computational alanine scan indicated that Tyr12 and Arg14 were likely key binding residues, given the Y12A and R14A mutations were the most unfavourable for binding free energy. MM/GBSA was then performed to calculate the effect of the Y12A and R14A mutations on the overall binding free energy of the peptide. The resulting binding free energies were-94.6 kcal/mol for Y12A and −103.4 kcal/mol for R14A. Compared to the −105.8 kcal/mol binding free energy calculated for SEQ ID NO. 1, the Y12A mutation significantly reduced binding affinity for PD-1.

Therefore, the Tyr12 residue was selected for further in silico mutagenesis with the goal of designing a null peptide to use as a control for in vitro experiments. When Tyr12 was mutated to each canonical amino acid, the most unfavourable substitutions at this position were Asp, Glu, and Gly. Subsequent MM/GBSA calculations demonstrated that the Y12E mutation significantly reduced binding affinity of the peptide, with a calculated binding free energy of −70.6 kcal/mol. The Y12E mutation was therefore selected to construct the MN_Null peptide (SEQ ID NO. 14).

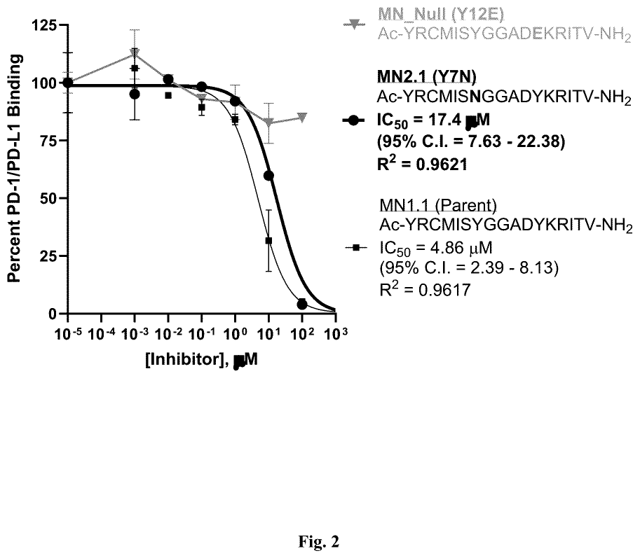

MN1.1 and MN_Null were then compared in vitro via Amplified Luminescent Proximity Homogeneous Assay (AlphaScreen), a bead-based biophysical screening technique used to measure PD-1/PD-L1 complex formation. AlphaScreen experiments showed that the parent peptide (MN1.1) was effective in blocking PD-1/PD-L1 complex formation, whereas the Y12E mutation eliminated the ability of MN_Null to block PD-1/PD-L1 complex formation, as predicted by the modeling data (FIG. 2).

Table 3 also indicates via computational alanine scanning that mutation Y12E of MN 1.1 significantly reduce activity. Y12E was computationally validated as a null mutation, as compared to Y7Q which tolerates mutation. Result is shown in Table 4, and FIG. 2.

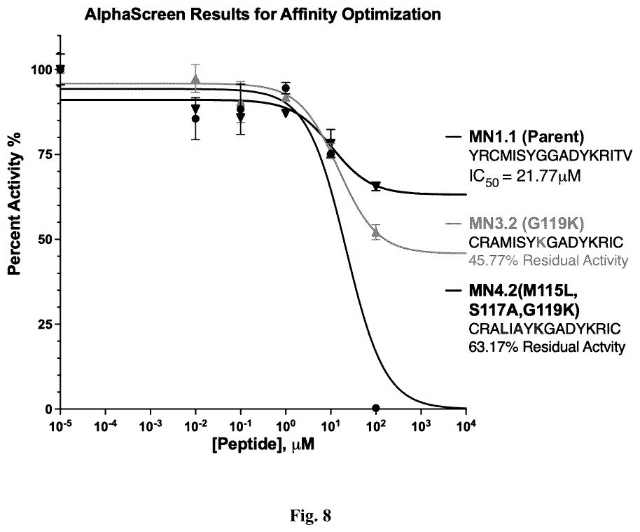

The next set of mutations that were attempted to improve the affinity of MN1.1 were identified a study reporting high affinity mutant PD-L176. Three of the reported high affinity mutations were within the region of PD-L1 that was used to design MN1.1. All three mutations were incorporated into the peptide sequence to create a new peptide, MN4.2 (SEQ ID NO. 8). One of the published mutations was predicted to introduce another hydrogen bond interaction with PD-1, and this one mutation was added to the β-loop region of the peptide to make MN3.2 (SEQ ID NO. 7). Peptides were tested via AlphaScreen as shown in FIG. 8.

The results from this round of AlphaScreening showed that these mutations worsened the affinity of MN1.1 for PD-1. This likely meant that these mutations were efficacious for improving affinity of the full-length protein, but not a short peptide sequence derived from native PD-L1.

Attempts to mutate MN1.1 with the goal of improving binding affinity were unsuccessful. Candidate peptides were generated with mutations that were informed by either our in silico data or reported in a recent study of high-affinity PD-L1 mutants6. However, all candidate peptides performed worse than MN1.1 when tested via AlphaScreen, with the majority having weak or no ability to PD-1/PD-L1 complex formation (Fig. S1). This suggested that the sequence of the PD-L1 interface used to design MN1.1 was likely not amenable to amino acid substitution and could possibly be the minimal region of PD-L1 critical for PD-1 binding.

Stabilization of the structure for in-vivo stability dictates that key substrate residues for trypsin and chymotrypsin like proteases, including Arg113, Tyr123, Lys124, and Arg125, would be difficult to mutate without losing potency, as demonstrated by the Y123E null mutant.

Because attempts to improve affinity of MN1.1 failed, modifications to improve the stability of MN1.1 were pursued. This included the addition of N-methyl groups on the peptide backbone for common protease targets to prevent degradation. The number of N-methyl groups that could be added to MN1.1 was limited due to the mechanism of peptide synthesis and only one N-methyl group was able to be successfully added to create peptide MN1.4 (N-methylArg124) (SEQ ID NO. 5). The sequences could be Acetyl-CRAMISYGGADYK-(N-Me-Arg)-IC-Amide, in which the N-terminal and C-terminal residues for a disulfide bond to cyclize the peptide.

MALDI data showed that the Met114 residue in MN1.4 became oxidized over time. While oxidation isn't as common of a modification in vivo, modelling data suggested oxidation of this residue might affect interactions with PD-1. Therefore, Met115 was substituted with a carbon analog, norleucine, which is structurally similar but contains a methylene group instead of the thioether group.

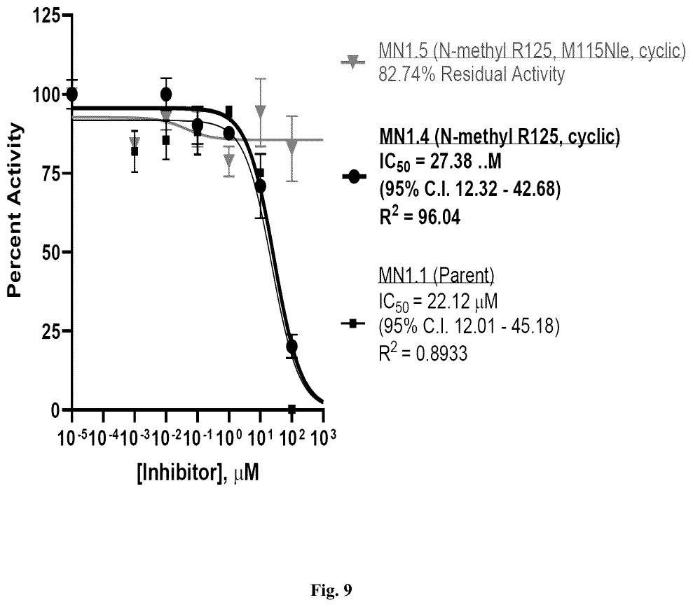

Mutation of Met115 to norleucine to create MN1.5, a common methionine isostere that is not subject to oxidation, could also be deleterious for activity. (SEQ ID NO. 6). An AlphaScreen experiment was run with the stabilized peptides to ensure the modifications did not worsen affinity. The Met115Nle mutation abolished the ability of MN1.1 to block PD-1/PD-L1 complex formation. This suggested the thioester group of Met114 formed important interactions with PD-1 and was important for peptide affinity. However, the N-methylation of Arg124 did not significantly impact efficacy, as shown in FIG. 9.

The results showed that N-methylation and the Y1C, C₃A, T16C mutations and V17 deletion required for cyclization did not significantly impact the activity of MN1.4 when compared to MN1.1.

Computational alanine scanning demonstrates residues 2, 4, and 11-14 of MN 1.1 reduce binding affinity if mutated.

TABLE 2
Computational alanine scanning of mutated MN 1.1

| Peptide | PD-L1 | Original | Mutated | Δ |
|---|---|---|---|---|
| Residue | Residue | AA | AA | Affinity |
| A:1 | 112 | TYR | ALA | −0.06 |
| A:2 | 113 | ARG | ALA | 16.53 |
| A:3 | 114 | CYS | ALA | 0.04 |
| A:4 | 115 | MET | ALA | 14.12 |
| A:5 | 116 | ILE | ALA | 0.1 |
| A:6 | 117 | SER | ALA | −0.27 |
| A:7 | 118 | TYR | ALA | −0.02 |
| A:8 | 119 | GLY | ALA | −0.06 |
| A:9 | 120 | GLY | ALA | −1.5 |
| A:10 | 121 | ALA | ALA | 0 |
| A:11 | 122 | ASP | ALA | 6.01 |
| A:12 | 123 | TYR | ALA | 24.04 |
| A:13 | 124 | LYS | ALA | 7.64 |
| A:14 | 125 | ARG | ALA | 25.39 |
| A:15 | 126 | ILE | ALA | 0.17 |
| A:16 | 127 | THR | ALA | −0.03 |
| A:17 | 128 | VAL | ALA | 0 |

TABLE 3
Computational alanine scanning of mutated
tyrosine at position 12 of MN 1.1

| Peptide | Original | Mutated | Δ |
|---|---|---|---|
| Residue | AA | AA | Affinity |
| A:12 | TYR | ARG | 3.3 |
| A:12 | TYR | PHE | 4.94 |
| A:12 | TYR | MET | 6.31 |
| A:12 | TYR | HIP | 10.07 |
| A:12 | TYR | LEU | 11.68 |
| A:12 | TYR | ILE | 12.25 |
| A:12 | TYR | GLN | 12.39 |
| A:12 | TYR | HID | 13.87 |
| A:12 | TYR | LYS | 14.22 |
| A:12 | TYR | HIE | 15.44 |
| A:12 | TYR | VAL | 16 |
| A:12 | TYR | TRP | 17.29 |
| A:12 | TYR | PRO | 19.83 |
| A:12 | TYR | CYS | 22.53 |
| A:12 | TYR | ASN | 23.5 |
| A:12 | TYR | ALA | 24.04 |
| A:12 | TYR | SER | 24.1 |
| A:12 | TYR | GLU | 25.13 |
| A:12 | TYR | GLY | 27.01 |
| A:12 | TYR | ASP | 33.42 |

TABLE 4
Computational validation of mutated MN 1.1

| Peptide | MMGBSA ΔG Bind |
|---|---|
| D11Q | −110.20 |
| D11A | −109.18 |
| MN1.1 (parent) | −105.77 |
| Y7Q | −105.43 |
| R14A | −103.41 |
| Y12A | −94.64 |
| Y12D | −74.63 |
| Y12E | −70.57 |
| D11Q Cyclic | −115.05 |
| MN1.1 Cyclic (parent) | −112.63 |
| Y7Q Cyclic | −110.88 |
| Y12E Cyclic | −64.23 |

However, the modelling did suggest that further truncation of the peptide on either the N- or C-terminal ends by one or two resides, respectively, mutation of the Cys114 residue, or peptide cyclization would be tolerated.

For this reason, stabilization of the structure was investigated via a one AA truncation on the N-terminus, 2AA truncation on the C-terminus, subsequent disulfide cyclization through added cysteine residues on the N- and C-terminus, and substitution of Arg125 with N-methyl Arg125 to protect this reside from degradation. This stabilized lead candidate is called SEQ ID NO. 5.

### Example 2: Validation of Binding of Optimized Candidates and Serum Stability of Lead Candidate

Given that many MN1.1 residues appeared crucial for PD-1 binding, modifications to improve peptide stability rather than affinity were pursued. Modifications included both head-to-tail cyclization and peptide backbone N-methylation, which have been shown to protect against protease degradation³⁵⁻³⁸.

In order to cyclize MN1.1 via a head-to-tail disulfide bond, several amino acid substitutions were introduced, and the peptide was truncated by removing Val17.

Flanking cysteine residues were introduced into the peptide sequence by substituting Tyr1 and Thr16 with cysteine, and Cys3 was mutated to alanine to prevent erroneous disulfide bond formation at this position. This cyclized peptide was named MN1.2 (SEQ ID NO. 4). In an embodiment, MN 1.2 have IC₅₀ values under 10 μM.

For further stabilization, an N-methyl group was added to the peptide backbone of MN1.2 to create peptide MN1.4 (N-methylR14) (SEQ ID NO. 5). This modification was expected to reduce protease cleavage at the K13-R14 peptide bond, given that lysine and arginine are native substrates for trypsin-like proteases³⁶⁻³⁸.

Following predicted mutations tolerated for increasing stability without sacrificing potency, a number of analog peptides were prepared and tested in the AlphaScreen model. As shown in FIG. 9, SEQ ID NO. 5 which incorporated only “allowed” stabilizing mutations as predicted by molecular modelling, did not lose potency as compared to the parent peptide, as shown in Table 5.

TABLE 5
Residual activity of peptides

| SEQ ID NO. | IC50 | Residual Activity |
|---|---|---|
| 2 | 22.12 μM | |
| | (95% C.I. 12.01-45.18) | |
| 5 | 27.38 μM | |
| | (95% C.I. 12.32-42.68) | |
| 6 | — | 82.74% |
| 7 | — | 45.77% |
| 8 | — | 62.87% |

However, peptides with unallowed mutations (SEQ ID NO. 6, M115Nle) lost activity. Furthermore, several mutations in the region of PD-L1 covered by MN 1.1 did not, in isolation, increase affinity of the peptide. MALDI MS was utilized to determine how stable the parent compound MN 1.1 was in serum, and what the major degradation products were.

Stability experiments were conducted with MN1.1, MN1.2, and MN1.4 to examine the impact of cyclization alone and any potential improvements to stability by the addition of N-methylation. Preliminary time course experiments were run for MN1.1 and MN1.4 to observe patterns of degradation and modification over the course of 6 hours via MALDI-TOF-MS.

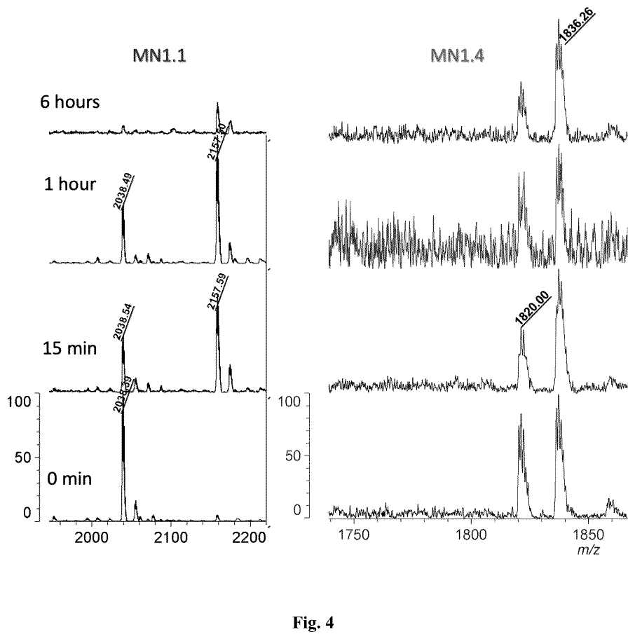

As shown in FIG. 4, MN1.1 was almost completely degraded after 6 hours. Interestingly, after 15 minutes, a new peak for MN1.1 appeared with a mass shift of +119 Da. This was consistent with a cysteinylation reaction between the cysteine of MN1.1 and a free cysteine in the serum39. This modification was not observed with MN1.4, given a C3A mutation was introduced to prevent the erroneous formation of the head-to-tail disulfide bond used to cyclize the peptide. Oxidation of the methionine was observed with MN1.4 (mass shift +16 Da), and both the oxidized and unoxidized forms of MN1.4 were still present after 6 hours in serum without significant degradation.

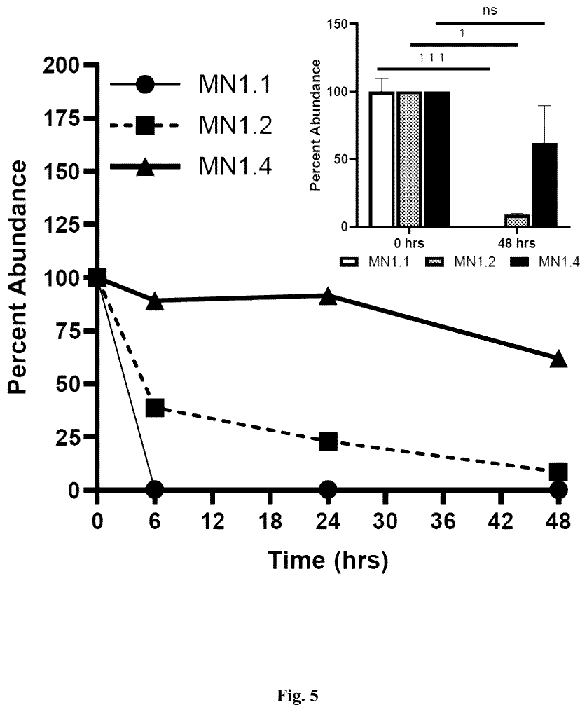

To quantify peptide levels and measure the stepwise improvements resulting from the introduced modifications, electrospray ionization (ESI) mass spectrometry was used to measure levels of MN1.1, MN1.2, and MN1.4 over the course of 48 hours. ESI-MS results showed stepwise improvements in stability with each additional modification, as shown in FIG. 5. Angiotensin I was spiked in and used for normalization. Consistent with the MALDI-TOF results, MN1.1 was completely degraded after 6 hours. MN1.2 was present at very low levels after 48 hours, and MN1.4 was not significantly degraded at 48 hours. These results demonstrated that the modifications made to the MN1.1 parent peptide were successful for improving peptide stability.

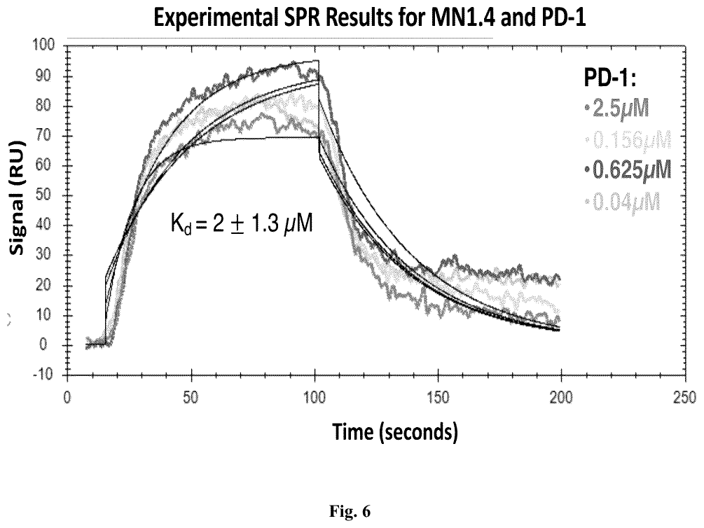

While oxidation is not a common type of post-translational modification in vivo⁴⁰, the Met4 residue of MN1.4 was replaced with a structural analog, norleucine, to prevent the oxidation observed via MALD-TOF-MS. However, the peptide created with the Met4Nle substitution, MN1.5, was not able to block PD-1/PD-L1 complex formation when tested via AlphaScreen, suggesting that the thioether of this Met residue could be important for PD-1 binding. Additionally, the affinity of MN1.4 for recombinant PD-1 was measured via surface plasmon resonance (FIG. 6). Interestingly, the measured K_{d} of MN1.4 was ˜2 μM, which was a similar order of magnitude as the K_{d} values reported for PD-1/PD-L1 in literature⁵⁻⁸.

As seen in FIG. 4 at time 0, MN 1.1 has one minor peak associated with oxidation of it's methionine residue. However, by 15 min incubation, about half of the peptide has been cysteinylated on residue C114. By 1 hr, the cysteinylated peptide is the major species, and by 6 hours the peptide has been virtually completely degraded.

Peptides were quantified via ESI MS and relatively abundances were compared at 6 hrs, 24 hrs, and 48 hrs for the parent MN1.1, a cyclized version of the MN2.1 (SEQ ID NO. 3), and the lead stabilized candidate SEQ ID NO. 5 is shown in FIG. 5.

Unlike the parent, which was gone by 6 hrs, the cyclized version of the parent persisted until roughly 48 hrs, by which point it was virtually completely degraded.

However, lead candidate SEQ ID NO. 5 retained roughly 75% of the original material by 48 hrs, leading to insignificant reduction. Therefore, SEQ ID NO. 5 was considering significantly stabilized and ready for in-vitro efficacy studies.

Therefore, as a stabilized mutant with limited variation from the wild-type sequence, we believed MN1.4 (SEQ ID NO. 5) was a compelling probe to investigate the minimal PD-L1 region necessary to trigger PD-1 signalling in a cell-based model.

### Example 3: In-vitro Efficacy of SEQ ID NO. 5

A co-culture model with OVCAR8 cells and human peripheral blood mononuclear cells (PBMCs) was designed to examine the immunomodulatory effects of MN1.4 treatment in the context of ovarian cancer. For this model, PBMCs and cancer cells were both stimulated prior to co-culture to promote expression of PD-1 and PD-L1, respectively.

To ensure the co-culture model was suitable for measuring changes in T cell responses after treatment, several rounds of optimization were completed before testing MN1.4. This included choosing the proper line to represent T cells in the MLR co-culture model and testing different stimulating conditions for the T cell line chosen.

Following stabilization, efficacy of the peptide having MN1.4 (SEQ ID NO. 5) was determined using a coculture model of human PBMCs, stimulated with CD3 and CD28 antibodies, with ovarian cancer line OVCAR8. Stimulation of the PBMCs causes them to release inflammatory cytokines including IL-2 and TNFα,

PD-1 agonist molecules should reduce the quantity of released inflammatory cytokines, while an antagonist of the interaction would increase the release of inflammatory cytokines.

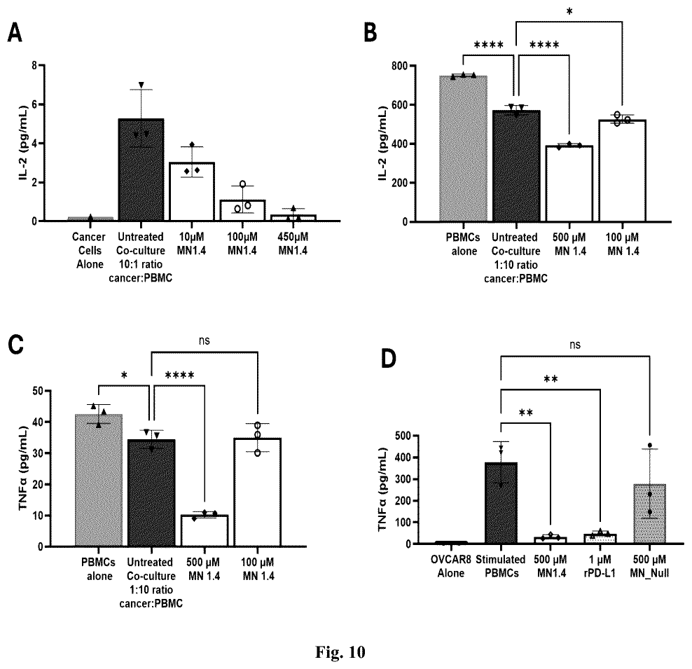

The results suggested MN1.4 enhanced the suppression of IL-2 secretion by T cells in a dose-dependent manner. However, the IL-2 concentrations detected in this trial were very low (<6 pg/mL), likely due to low PBMC concentrations resulting from the use of a 10:1 ratio of excess OVCAR8 cells in this experiment. (FIG. 10A)

Therefore, co-culture experiments were repeated using a 1:10 ratio of OVCAR8: PBMCs to increase the dynamic range of the assay and improve statistical confidence. In addition, other markers of T cell activation, TNFα, were measured via traditional ELISA to ensure the effects of MN1.4 were monitored using multiple outputs. Consistent with the results from 10:1 ratio co-culture, the same pattern of T cell suppression was observed when IL-2 and TNFα, levels were measured via ELISA for co-cultures in 1:10 ratio, as shown in (FIG. 10B, C). Furthermore, experiments with MN_Null showed less immunosuppressive activity as compared to MN1.4. (FIG. 10D)

To investigate whether the biological activity of MN1.4 was similar to that of full-length PD-L1, T cell responses to treatment with MN1.4 and recombinant, full-length PD-L1 (rPD-L1) were compared using the co-culture model. As shown in FIG. 10D, treatment with MN1.4 and rPD-L1 significantly decreased IL-2 andTNFα, levels compared to the untreated co-culture control. Additionally, there was not a significant difference in cytokine levels between the MN1.4 and rPD-L1 treatment groups, although the potency of the peptide and the recombinant protein differed.

Upon addition of increasing amounts of SEQ ID NO. 5, secretion of IL-2 reduced in a dose dependent manner, as shown in FIG. 10A.

To validate this response, the ratio of PBMCs to cancer cells was inverted, a new female PBMC donor was selected, and different cytokines were measured: IL-2 and TNFα.

In each case, the released cytokines were reduced in a dose-dependent manner as shown in FIG. 10B, C. Moreover, as expected for cytokine IL-2 (T-cell specific), adding the PD-L1+ ovarian cells reduced the released IL-2 as compared to stimulated PBMCs alone, further validating the model. Finally, two additional controls were examined: recombinant PD-L1, as well as addition of the MN_Null (SEQ ID NO. 15), as shown in FIG. 10D. Here, while MN 1.4 (SEQ ID NO. 5) is less potent than recombinant PD-L1, to elicits the same effect, while the MN_Null peptide with little affinity for PD-1 has little immunosuppressive effect.

Based on the totality of the data, SEQ ID NO. 5 represents a simple, stable PD-1 binding peptide with immunosuppressive activity that has the potential for further development as a clinical PD-1 agonist.

### DISCUSSION

In an embodiment, we report the identification of essential PD-1 binding residues of a small peptide derived from the PD-L1 interface region, and the development of an optimized peptide, MN1.4 (SEQ ID NO. 5), which underwent functional characterization in a cell-based model. MN1.4 (SEQ ID NO. 5) was designed with variation from the native PD-L1 sequence, with the exception of substitutions required for cyclization near the terminal ends of the peptide (Tyr1, Cys3, Thr16, Val17). The results of computational alanine scanning, and in vitro experiments suggested that mutations were not tolerated between Met4-Arg14, including mutations to alanine or simple changes to sidechain chemistry, indicating that this region could be the minimal region of PD-L1 critical for PD-1 binding.

When the biological activity of MN1.4 was tested in a cell-based assay, MN1.4 recapitulated the biological effects of full-length PD-L1, functioning as an activator of PD-1 signaling. This finding has several interesting implications, including that MN1.4 likely contains the biologically active sequence of PD-L1 required to trigger PD-1 signalling and suppress T cell responses. Additionally, given MN1.4 is a synthetic peptide, it lacks the same post-translational modifications (PTMs) as native PD-L1. Glycosylation has been shown to be very important for this complex by regulating the stability of cell surface PD-1/PD-L1 and modulating immunosuppressive properties of cell surface PD-L117.47. However, the PD-L1 sequence used to design MN1.4 did not contain any of the canonical N-glycosylation sites of PD-L1, suggesting glycosylation of the peptide was not necessary for initiation of PD-1 signaling in this model. However, further studies into the role of PTMs in the context of peptidomimetics/target protein interactions may uncover new opportunities for peptide optimization via PTMs.

Treatment was a high dose of MN1.4 was shown to decrease IL-2 and TNFα secretion in the co-culture model. The therapeutic anti-PD-1 antibody, Pembrolizumab, did not have this effect. This suggested that MN1.4 may act as an agonist for PD-1, rather than an inhibitor. To investigate this, recombinant full-length PD-L1 was tested as a control in the co-culture model. MN1.4 and recombinant PD-L1 were both shown to significantly decrease cytokine levels, and the immunosuppressive effects of MN1.4 and recombinant PD-L1 were not significantly different from each other when measured via IL-2 and TNFα, =ELISA. These results supported the new hypothesis that MN1.4 is an agonist for PD-1 instead of an inhibitor.

No small peptide agonists of PD-1 have been reported to date, and while recent studies have examined PD-1 agonist antibodies or fusion proteins mimicking PD-L1, few studies have also focused on functional characterization of the native PD-L1 interface sequence to determine the region essential for triggering PD-1 signaling41-43. While studies have published small targeting peptides that mimic the PD-L1 interface, these studies have done so in the context of PD-1 antagonism12.14.42. Previous studies have either introduced significant modifications to the native PD-L1 interface sequence or did not include biological characterization experiments, such as testing in a cell-based model. In this work, we identified and characterized the active and functional region of PD-L1 required for binding and initiation of PD-1 signalling. To our knowledge, this is the first study to compare the biological function of this PD-L1 interface region to the function of native, full-length PD-L1.

Future directions include comparing the affinity of peptides built from the MN1.4 scaffold with similar sequences but differing structural rigidities to enable a more precise determination of the affinity required to transition a ligand's biological effect from PD-1 agonism to antagonism. While this hypothesis was not able to be fully explored in this work, we believe ligand affinity for PD-1 could possibly be the delineating factor between agonists and antagonists, in which molecules with affinities in the low micromolar range, similar to MN1.4 and native PD-L1, could function as agonists, whereas those with higher affinities could function as antagonists. The lowest affinity reported for a protein antagonist of PD-1 is 750 nM compared to 17 μM of native PD-L16; suggesting that a 20-fold improvement in affinity over the native protein is sufficient to transition to antagonism. The idea that ligand affinity could potentially determine agonism versus antagonism of PD-1 signalling has interesting implications for the design of PD-1 agonists.

Most drug discovery campaigns begin with affinity and specificity optimization, and biological efficacy is rarely evaluated for compounds that do not reach nanomolar affinities. However, this approach may prove detrimental in the development of agonists for PD-1, where prioritization of lower affinity compounds may be required. After further optimization to increase the potency of MN1.4 while maintaining agonism, the resulting optimized peptides could be used in pre-clinical pharmacokinetic/pharmacodynamics models to better evaluate dosing and off-target effects as a function of affinity before moving to disease models. Peptides optimized from the MN1.4 scaffold could be used as immunosuppressants for treating autoimmune diseases, such as those caused by conventional mAb-based immunotherapies, in addition to treating several common diseases shown to have insufficient PD-1/PD-L1 signalling, such as diabetes and arthritis 19.20. After completion of peptide optimization and dosing studies, efficacy testing should be performed in mouse models of diseases caused by deficient PD-1 signalling, such as rheumatoid arthritis, Crohn's diseases, or inflammatory bowel disease in order to fully understand the immunomodulatory effects of a peptide PD-1 agonist. Such an optimized compound could be a first-in-class immunosuppressant without many of the associated downsides of steroids and other standards of care.

### Methodology

### Computational Alanine Scanning

Computational alanine scanning was performed using the Schrödinger Bioluminate Suite Residue Scanning feature in Schrödinger Maestro version 2020.2 (New York, NY, USA). The published crystal structure of hPD-1/PD-L1 (PDB: 4ZQK18) was first modified to include full-length PD-1 and only the PD-L1 residues corresponding to MN1.1 (residues 112-128). The MN1.1 residues were renumbered from 1-17 for clarity, and this structure was then used for computational alanine scanning. Changes in the binding free energy of each residue in the MN1.1 region of PD-L1 was calculated after mutation to alanine to identify residues with high and low affinity for PD-1. To assess the effect of selected alanine mutations on the binding free energy of the overall peptide, Mechanics Generalized Born and Surface Area (MM/GBSA) calculations were performed in Schrödinger Maestro. Residues of interest identified via computational alanine scan underwent further in silico mutagenesis to each canonical amino acid to predict mutations that would either reduce or enhance binding to PD-1. MM/GBSA calculations were then performed for selected mutations to calculate binding free energy changes at the peptide level. All parameter, starting coordinate PDBs, and script files are provided on a public OSF page.

### AlphaScreening Experiments

Peptides were screened using the PerkinElmer PD-1/PD-L1 (human) AlphaLISA Binding Kit (Waltham, MA, USA, Cat. No. AL356HV) according to the manufacturer's instructions. MN1.1, MN_Null, MN2.1, MN3.2, MN4.2, MN1.4, and MN1.5 were purchased from Peptide 2.0. Sequences for all peptides utilized in this study are provided in Table S1. Briefly, peptides were resuspended in assay buffer with 4% dimethyl sulfoxide (DMSO) to make 4× stock solutions of 400 μM. The biotinylated PD-1 and His-tagged PD-L1 included in the kit were then resuspended in assay buffer for 4× stock concentrations of 20 nM. Lastly, the 4× mixture of anti-His Acceptor beads and streptavidin Donor beads were prepared in the dark. Peptides were then added in duplicates of 10 μL to a white 96-well ½-AreaPlate (PerkinElmer, Cat. No. 6005560) at 7 different concentrations from 1 nM to 100 μM. Equal volumes of the biotinylated PD-1 and His-tagged PD-L1 included in the kit were added to the plate (final concentration 5 nM), followed by the mixture of Acceptor and Donor beads (final concentrations 10 μg/mL and 20 μg/mL, respectively). The plate was incubated in the dark for 90 minutes at room temperature and luminescence was measured using a Tecan Spark multimode plate reader (Männedorf, Switzerland). For data analysis, a control with beads alone was subtracted from each well for background correction. Data analysis was conducted using GraphPad Prism version 9 (La Jolla, CA, USA).

### Serum Stability Experiments

Peptide stocks for MN1.1, MN1.2, and MN1.4 (Peptide 2.0) were prepared at a concentration of 10 μg/μL in DMSO. Human serum was diluted 1:4 in Gibco RPMI 1640 media (ThermoFisher Scientific, Waltham, MA, USA, Cat. No. 11-875-093) for a total of 1 mL and incubated at 37° C. for 15 minutes prior to time course. Next, 1 μL of the peptide stock was added to warmed serum for a final concentration of 10 g/mL in the master mix. The rest of the time course was performed at room temperature. For each time point, aliquots of 100 μL were removed from the master mix and added to 200 μL of 3% trifluoroacetic acid. Samples were incubated on ice for 15 minutes before centrifugation at 16,000×g for 2.5 minutes. Supernatants were transferred to a clean tube and prepared for desalting via C18 column by adding 100 μL of sample buffer (20% acetonitrile, 2% trifluoroacetic acid) to supernatants before storage at—20° C. overnight. Samples were then processed through Pierce C18 spin columns (ThermoFisher Scientific, Cat. No. 89870) according to the manufacturer's instructions. Desalted samples were dried under nitrogen and stored at −20° C. until mass spectrometry analysis.

For MALDI-TOF-MS analysis, serum stability samples were reconstituted in 200 μL of 0.1% formic acid. Samples were then diluted 1:10 in a MALDI matrix containing a 50 fmol/μL solution of Angiotensin I as a control. Samples were spotted onto the plate at a volume of 1 μL and were run on a Axima Performance MALDI TOF/TOF mass spectrometer (Shimadzu Scientific Instruments, Columbia, MD, USA). Data was visualized with Axima Launchpad 2.8 software (Shimadzu Scientific Instruments).

For ESI-MS analysis, serum stability samples were run on an Orbitrap Exploris 480 (ThermoFisher Scientific). Samples were spiked with 25 fmol Angiotensin I as an internal control. Peptides were separated using a reversed-phase PepMap RSLC 75 μm inner diameter×15 cm long with 2 μm particle size, C18 resin LC column (ThermoFisher Scientific, Cat. No. 164534). Mobile phase A consisted of 0.1% aqueous formic acid and mobile phase B consisted of 0.1% formic acid in 80% acetonitrile. After sample injection (2 μL), the peptides were eluted by using a linear gradient from 5 to 50% B over 30 min and ramping to 100% B for an additional 2 minutes. The flow rate was set at 300 nl/min. The mass spectrometer was operated in a data-dependent mode in which one full MS scan (60,000 resolving power) from 300 m/z to 1800 m/z using quadrupole isolation was followed by MS/MS scans in which the most abundant molecular ions were dynamically selected and fragmented by higher energy collisional dissociation using a normalized collision energy of 27%. ThermoFisher's Xcalibur 4.0 Data System was used for peptide quantification. Qual Browser was used to determine the retention time and most abundant charge state for each peptide. This information was used to create a processing workflow in the Processing Setup application of Xcalibur. In the Sequence Setup application, the processing workflow was paired with the corresponding instrument method used for each peptide (see settings above). The samples were batch reprocessed for quantification via peak area. Peptide abundances were normalized based on Angiotensin I abundance and quantified relative to initial abundance.

### Surface Plasmon Resonance

Biotinylated (N-terminus modified) MN1.4 (Peptide 2.0, Chantilly, VA, USA) was immobilized at a concentration of 100 μM on Nicoya streptavidin-conjugated gold sensor chips (Ontario, Canada, Cat. No. SEN-AU-100-10) and inserted into the flow chamber of a benchtop Nicoya OpenSPR 2-channel device. Dilutions of recombinant human PD-1 from R&D Systems (Minneapolis, MN, USA, Cat. No. 8986-PD-100) were prepared in sterile PBS and allowed to flow over the chip at a flow rate of 50 μL/min. Traces were evaluated using the TraceDrawer software (Uppsala, Sweden). Presented Kd value is the average of two independent trials.

### Cell Culture Conditions

OVCAR8 cells, generously provided by the Gottesman Lab (National Institutes of Health, Bethesda, MD, USA), and MDA-MB-231 cells (American Type Culture Collection, Rockville, MD, USA) were cultured in Gibco RPMI 1640 media (ThermoFisher Scientific, Cat. No. 11-875-093) supplemented with 10% Gibco fetal bovine serum (ThermoFisher Scientific, Cat. No. 10437028), and 1% penicillin/streptomycin (10,000 U/mL pen, 10 mg/mL strcp) from MilliporeSigma (Burlington, MA, USA, Cat. No. P4333). Cells were cultured at 37° C. and 5% CO2. Passage number was kept below 3 for co-culture assay. Human peripheral blood mononuclear cells were purchased frozen from STEMCELL Technologies (Vancouver, Canada, Cat. No. 70025.2) from two female donors. PBMCs were thawed according to manufacturer's instructions one day before co-culture and cultured in the same media and conditions as cancer cell lines.

### Co-Culture Conditions

For co-culture experiments with excess OVCAR8 ratios, OVCAR8 cells were seeded at a density of 2×105 cells/mL on a 6-well plate and allowed to adhere overnight. The next day, cells were stimulated with 100 ng/mL IFNγ (R&D Systems, Cat. No. 285-IF-100), and two vials of human PBMCs were thawed according to manufacturer's instructions. On Day 3, concentrations of PBMCs and OVCAR8 cells (harvested from a control well) were measured using the QuadCount Automated Cell Counter (Accuris Instruments). The concentration of PBMCs required for a 10:1 OVCAR8:PBMC ratio was calculated from the OVCAR8 concentration. PBMCs were then seeded on a separate 6-well plate (1 mL/well) and treated with MN1.4 (450 μM, 100 μM, or 10 μM) from Lifetein (Somerset, NJ, USA) for 30 minutes prior to co-culture. After 30 minutes, media was removed from OVCAR8 wells and replaced with 1 mL of treated PBMCs. PBMCs were stimulated 30 minutes after co-culture with 5 μg/mL of anti-CD3 (Cat. No. 555329) and 5 μg/mL of anti-CD28 (Cat. No. 567117) from BD Biosciences (San Jose, CA, USA). After 24 hours, the supernatants were collected from each well, and centrifuged at 10,000×g for 5 minutes to remove any remaining cells.

For co-culture experiments with excess PBMCs ratios, OVCAR8 cells were seeded at a density of 2×104 cells/mL on a 24-well plate and allowed to adhere overnight. The next day, cells were stimulated with 100 ng/mL IFNγ and two vials of human PBMCs were thawed. On Day 3, concentrations of PBMCs and OVCAR8 cells (harvested from a control well) were measured using the QuadCount Automated Cell Counter. The concentration of PBMCs required for a 1:10 OVCAR8:PBMC ratio was calculated from the OVCAR8 concentration. PBMCs were plated on a separate 24-well plate (1 mL/well) and stimulated with 5 μg/mL of anti-CD3 and 5 μg/mL of anti-CD28 for 30 minutes before drug treatment. PBMCs were then treated with MN1.4 (500 μM, 100 μM, or 10 μM) from Peptide 2.0 or 1 μM recombinant human PD-L1 (R&D systems, Cat. No. 156-B7-100) for 30 minutes prior to co-culture. After 30 minutes, media was removed from OVCAR8 wells without treatment, and replaced with 1 mL of treated PBMCs. After 24 hours, the supernatants were collected from each well, and centrifuged at 10,000×g for 5 minutes to remove any remaining cells.

### Co-Culture ELISAs and Data Analysis

For experiments analyzed via IL-2 PCR-ELISA, the levels of IL-2 in the collected supernatants were measured using the IL-2 Human ProQuantum Immunoassay Kit (ThermoFisher Scientific, Cat. No. A35603). Briefly, samples from co-culture experiments with excess OVCAR8 ratios were diluted 1:10 in assay buffer, and samples from co-culture experiments with excess PBMC ratios were diluted 1:5 in assay buffer. Next, 10 μL of the diluted samples were mixed with 10 μL of an IL-2 antibody-oligonucleotide conjugate mixture and added to a 96-well working plate. Samples with excess OVCAR8 ratios were allowed to incubate overnight at 4° C. Samples with excess PBMCs were allowed to incubate for 1 hour at room temperature. Next, 80 μL of qPCR reaction mixture containing master mix and DNA ligase was added to each well. The plate was centrifuged at 2,500×g for 2 minutes. Samples were then added in triplicate (20 μL/well) to a 96-well PCR plate (ThermoFisher Scientific). PCR was run according to manufacturer's instructions using the QuantStudio 7 Pro Real-Time PCR System equipped with a 96-well, 0.2 mL block from ThermoFisher Scientific.

Remaining samples from experiments with excess PBMC ratios were analyzed using TNFα (Cat. No. 430204) ELISA MAX Deluxe Sets (BioLegend, San Diego, CA, USA) according to manufacturer's instructions. Briefly, 96-well Nunc™ MaxiSorp™ ELISA Plates (BioLegend, Cat. No. 423501) were coated with capture antibodies for TNFα and incubated overnight at 4° C. The next day, the plate was washed 4× with 1×PBS+0.05% Tween-20 (PBS-T). To block non-specific binding, 200 μL of assay dilutant buffer was added to each well and the plate was incubated at room temperature for 1 hour on a shaker. The plate was washed as before, and 100 μL of the samples or standards were added to each well in triplicate and incubated at room temperature for 2 hours on a shaker. The plate was washed 4× and 100 μL of detection antibody for TNFα was added to each well, and the plate was incubated at room temperature for 1 hour on a shaker. The plate was washed 4× and 100 μL of Avidin-HRP was added to each well, and the plate was incubated at room temperature for 30 minutes on a shaker. For the final wash step, the plate was washed 5× with ˜1 minute/wash to reduce background. Next, 100 μL of freshly mixed TMB (3,3′,5,5′-Tetramethylbenzidine) substrate solution was added to each well and the plate was incubated in the dark for 15 minutes. The reaction was then stopped by adding 100 μL of stop solution (2N H2SO4) to each well. Absorbance at 450 nm and 570 nm was measured using a Tecan Spark multimode plate reader. For data analysis, the absorbance at 570 nm was subtracted from the absorbance at 450 nm.

GraphPad Prism version 9 was used to analyze results from the IL-2 and TNFα, ELISA experiments. Nonlinear regression was performed to generate the standard curve for each assay and to interpolate sample concentrations. For significance testing, ordinary one-way ANOVA with Tukey correction for multiple comparisons was used to compare the treatment conditions to the untreated co-culture control. A p-value of <0.05 was chosen as the significance cut-off. Symbols for significance in all figures correlate to the following p-values: ns (p>0.05), *(p≤0.05), **(p ≤0.01), ***(p≤0.001), and ****(p≤0.0001). All bars represent average+/−SEM. To compare between markers, the values for each treatment condition were normalized to the untreated control.

### REFERENCES

All references, including granted patents and patent application publications, referred herein are incorporated herein by reference in their entirety.
(1) Qin, W.; Hu, L.; Zhang, X.; Jiang, S.; Li, J.; Zhang, Z.; Wang, X. The Diverse Function of PD-1/PD-L Pathway Beyond Cancer. Front. Immunol. 2019, 10, 2298. https://doi.org/10.3389/fimmu.2019.02298.
(2) Sharpe, A. H.; Pauken, K. E. The Diverse Functions of the PD1 Inhibitory Pathway. Nat Rev Immunol 2018, 18 (3), 153-167. https://doi.org/10.1038/nri.2017.108.
(3) Wang, J.; Yoshida, T.; Nakaki, F.; Hiai, H.; Okazaki, T.; Honjo, T. Establishment of NOD-Pdcd1−/−Mice as an Efficient Animal Model of Type I Diabetes. Proceedings of the National Academy of Sciences 2005, 102 (33), 11823-11828. https://doi.org/10.1073/pnas.0505497102.
(4) Reynoso, E. D.; Elpek, K. G.; Francisco, L.; Bronson, R.; Bellemare-Pelletier, A.; Sharpe, A. H.; Freeman, G. J.; Turley, S. J. Intestinal Tolerance Is Converted to Autoimmune Enteritis upon PD-1 Ligand Blockade. J Immunol 2009, 182 (4), 2102-2112. https://doi.org/10.4049/jimmunol.0802769.
(5) Bojko, M.; Węgrzyn, K.; Sikorska, E.; Kocikowski, M.; Parys, M.; Battin, C.; Steinberger, P.; Kogut, M. M.; Winnicki, M.; Sieradzan, A. K.; Spodzieja, M.; Rodziewicz-Motowidło, S. Design, Synthesis and Biological Evaluation of PD-1 Derived Peptides as Inhibitors of PD-1/PD-L1 Complex Formation for Cancer Therapy. Bioorganic Chemistry 2022, 128, 106047. https://doi.org/10.1016/j.bioorg.2022.106047.
(6) Liang, Z.; Tian, Y.; Cai, W.; Weng, Z.; Li, Y.; Zhang, H.; Bao, Y.; Li, Y. High-Affinity Human PD-L1 Variants Attenuate the Suppression of T Cell Activation. Oncotarget 2017, 8 (51), 88360-88375. https://doi.org/10.18632/oncotarget.21729.
(7) Li, K.; Cheng, X.; Tilevik, A.; Davis, S. J.; Zhu, C. In Situ and in Silico Kinetic Analyses of Programmed Cell Death-1 (PD-1) Receptor, Programmed Cell Death Ligands, and B7-1 Protein Interaction Network. J Biol Chem 2017, 292 (16), 6799-6809. https://doi.org/10.1074/jbc.M116.763888.
(8) Cheng, X.; Veverka, V.; Radhakrishnan, A.; Waters, L. C.; Muskett, F. W.; Morgan, S. H.; Huo, J.; Yu, C.; Evans, E. J.; Leslie, A. J.; Griffiths, M.; Stubberfield, C.; Griffin, R.; Henry, A. J.; Jansson, A.; Ladbury, J. E.; Ikemizu, S.; Carr, M. D.; Davis, S. J. Structure and Interactions of the Human Programmed Cell Death 1 Receptor. J Biol Chem 2013, 288 (17), 11771-11785. https://doi.org/10.1074/jbc.M112.448126.
(9) Fessas, P.; Lec, H.; Ikemizu, S.; Janowitz, T. A Molecular and Preclinical Comparison of the PD-1-Targeted T-Cell Checkpoint Inhibitors Nivolumab and Pembrolizumab. Semin Oncol 2017, 44 (2), 136-140. https://doi.org/10.1053/j.seminoncol.2017.06.002.
(10) Gong, B.; Kiyotani, K.; Sakata, S.; Nagano, S.; Kumchara, S.; Baba, S.; Besse, B.; Yanagitani, N.; Friboulet, L.; Nishio, M.; Takeuchi, K.; Kawamoto, H.; Fujita, N.; Katayama, R. Secreted PD-L1 Variants Mediate Resistance to PD-L1 Blockade Therapy in Non-Small Cell Lung Cancer. The Journal of Experimental Medicine 2019, 216 (4), 982-1000. https://doi.org/10.1084/jem.20180870.
(11) Mahoney, K. M.; Shukla, S. A.; Patsoukis, N.; Chaudhri, A.; Browne, E. P.; Arazi, A.; Eisenhaure, T. M.; Pendergraft, W. F.; Hua, P.; Pham, H. C.; Bu, X.; Zhu, B.; Hacohen, N.; Fritsch, E. F.; Boussiotis, V. A.; Wu, C. J.; Freeman, G. J. A Secreted PD-L1 Splice Variant That Covalently Dimerizes and Mediates Immunosuppression. Cancer Immunol Immunother 2019, 68 (3), 421-432. https://doi.org/10.1007/s00262-018-2282-1.
(12) Zhou, J.; Mahoney, K. M.; Giobbie-Hurder, A.; Zhao, F.; Lec, S.; Liao, X.; Rodig, S.; Li, J.; Wu, X.; Butterfield, L. H.; Piesche, M.; Manos, M. P.; Eastman, L. M.; Dranoff, G.; Freeman, G. J.; Hodi, F. S. Soluble PD-L1 as a Biomarker in Malignant Melanoma Treated with Checkpoint Blockade. Cancer Immunol Res 2017, 5 (6), 480-492. https://doi.org/10.1158/2326-6066.CIR-16-0329.
(13) Ng, K. W.; Attig, J.; Young, G. R.; Ottina, E.; Papamichos, S. I.; Kotsianidis, I.; Kassiotis, G. Soluble PD-L1 Generated by Endogenous Retroelement Exaptation Is a Receptor Antagonist. eLife 2019, 8, c50256. https://doi.org/10.7554/eLife.50256.
(14) Abbas, A.; Lin, B.; Liu, C.; Morshed, A.; Hu, J.; Xu, H. Design and Synthesis of A PD-1 Binding Peptide and Evaluation of Its Anti-Tumor Activity. IJMS 2019, 20 (3), 572. https://doi.org/10.3390/ijms20030572.
(15) Zhou, K.; Lu, J.; Yin, X.; Xu, H.; Li, L.; Ma, B. Structure-Based Derivation and Intramolecular Cyclization of Peptide Inhibitors from PD-1/PD-L1 Complex Interface as Immune Checkpoint Blockade for Breast Cancer Immunotherapy. Biophysical Chemistry 2019, 253, 106213. https://doi.org/10.1016/j.bpc.2019.106213.
(16) Li, C.-W.; Lim, S.-O.; Chung, E. M.; Kim, Y.-S.; Park, A. H.; Yao, J.; Cha, J.-H.; Xia, W.; Chan, L.-C.; Kim, T.; Chang, S.-S.; Lec, H.-H.; Chou, C.-K.; Liu, Y.-L.; Ych, H.-C.; Perillo, E. P.; Dunn, A. K.; Kuo, C.-W.; Khoo, K.-H.; Hsu, J. L.; Wu, Y.; Hsu, J.-M.; Yamaguchi, H.; Huang, T.-H.; Sahin, A. A.; Hortobagyi, G. N.; Yoo, S. S.; Hung, M.-C. Eradication of Triple-Negative Breast Cancer Cells by Targeting Glycosylated PD-L1. Cancer Cell 2018, 33 (2), 187-201.c10. https://doi.org/10.1016/j.ccell.2018.01.009.
(17) Li, C.-W.; Lim, S.-O.; Xia, W.; Lec, H.-H.; Chan, L.-C.; Kuo, C.-W.; Khoo, K.-H.; Chang, S.-S.; Cha, J.-H.; Kim, T.; Hsu, J. L.; Wu, Y.; Hsu, J.-M.; Yamaguchi, H.; Ding, Q.; Wang, Y.; Yao, J.; Lec, C.-C.; Wu, H.-J.; Sahin, A. A.; Allison, J. P.; Yu, D.; Hortobagyi, G. N.; Hung, M.-C. Glycosylation and Stabilization of Programmed Death Ligand-1 Suppresses T-Cell Activity. Nat Commun 2016, 7, 12632. https://doi.org/10.1038/ncomms12632.
(18) Zak, K. M.; Kitel, R.; Przetocka, S.; Golik, P.; Guzik, K.; Musiclak, B.; Dömling, A.; Dubin, G.; Holak, T. A. Structure of the Complex of Human Programmed Death 1, PD-1, and Its Ligand PD-L1. Structure 2015, 23 (12), 2341-2348. https://doi.org/10.1016/j.str.2015.09.010.
(19) Falcone, M.; Fousteri, G. Role of the PD-1/PD-L1 Dyad in the Maintenance of Pancreatic Immune Tolerance for Prevention of Type 1 Diabetes. Front. Endocrinol. 2020, 11, 569. https://doi.org/10.3389/fendo.2020.00569.
(20) Canavan, M.; Floudas, A.; Veale, D. J.; Fearon, U. The PD-1: PD-L1 Axis in Inflammatory Arthritis. BMC Rheumatol 2021, 5 (1), 1. https://doi.org/10.1186/s41927-020-00171-2.
(21) Haymond, A.; Dey, D.; Carter, R.; Dailing, A.; Nara, V.; Nara, P.; Venkatayogi, S.; Paige, M.; Liotta, L.; Luchini, A. Protein Painting, an Optimized MS-Based Technique, Reveals Functionally Relevant Interfaces of the PD-1/PD-L1 Complex and the YAP2/ZO-1 Complex. J. Biol. Chem. 2019, 294 (29), 11180-11198. https://doi.org/10.1074/jbc.RA118.007310.
(22) Kozakov, D.; Brenke, R.; Comeau, S. R.; Vajda, S. PIPER: An FFT-Based Protein Docking Program with Pairwise Potentials. Proteins 2006, 65 (2), 392-406. https://doi.org/10.1002/prot.21117.
(23) Abraham, M. J.; Murtola, T.; Schulz, R.; Páll, S.; Smith, J. C.; Hess, B.; Lindahl, E. GROMACS: High Performance Molecular Simulations through Multi-Level Parallelism from Laptops to Supercomputers. SoftwareX 2015, 1-2, 19-25. https://doi.org/10.1016/j.softx.2015.06.001.
(24) Huang, J.; Rauscher, S.; Nawrocki, G.; Ran, T.; Feig, M.; de Groot, B. L.; Grubmüller, H.; MacKerell, A. D. CHARMM36m: An Improved Force Field for Folded and Intrinsically Disordered Proteins. Nat Methods 2017, 14 (1), 71-73. https://doi.org/10.1038/nmeth.4067.
(25) Bussi, G.; Donadio, D.; Parrinello, M. Canonical Sampling through Velocity Rescaling. The Journal of Chemical Physics 2007, 126 (1), 014101. https://doi.org/10.1063/1.2408420.
(26) Parrinello, M.; Rahman, A. Polymorphic Transitions in Single Crystals: A New Molecular Dynamics Method. Journal of Applied Physics 1981, 52 (12), 7182-7190. https://doi.org/10.1063/1.328693.
(27) Essmann, U.; Perera, L.; Berkowitz, M. L.; Darden, T.; Lee, H.; Pedersen, L. G. A Smooth Particle Mesh Ewald Method. The Journal of Chemical Physics 1995, 103 (19), 8577-8593. https://doi.org/10.1063/1.470117.
(28) Verlet, L. Computer “Experiments” on Classical Fluids. I. Thermodynamical Properties of Lennard-Jones Molecules. Phys. Rev. 1967, 159 (1), 98-103. https://doi.org/10.1103/PhysRev.159.98.
(29) Hess, B. P-LINCS: A Parallel Linear Constraint Solver for Molecular Simulation. J. Chem. Theory Comput. 2008, 4 (1), 116-122. https://doi.org/10.1021/ct700200b.
(30) Miles, A. J.; Ramalli, S. G.; Wallace, B. A. DichroWeb, a Website for Calculating Protein Secondary Structure from Circular Dichroism Spectroscopic Data. Protein Science 2022, 31 (1), 37-46. https://doi.org/10.1002/pro.4153.
(31) Johnson, W. C. Analyzing Protein Circular Dichroism Spectra for Accurate Secondary Structures. Proteins 1999, 35 (3), 307-312.
(32) Abdul-Gader, A.; Miles, A. J.; Wallace, B. A. A Reference Dataset for the Analyses of Membrane Protein Secondary Structures and Transmembrane Residues Using Circular Dichroism Spectroscopy. Bioinformatics 2011, 27 (12), 1630-1636. https://doi.org/10.1093/bioinformatics/btr234.
(33) Luchini, A.; Espina, V.; Liotta, L. A. Protein Painting Reveals Solvent-Excluded Drug Targets Hidden within Native Protein-Protein Interfaces. Nature Communications 2014, 5 (1), 1-12. https://doi.org/10.1038/ncomms5413.
(34) Dong, Q.; Yan, X.; Kilpatrick, L. E.; Liang, Y.; Mirokhin, Y. A.; Roth, J. S.; Rudnick, P. A.; Stein, S. E. Tandem Mass Spectral Libraries of Peptides in Digests of Individual Proteins: Human Serum Albumin (HSA). Molecular & Cellular Proteomics 2014, 13 (9), 2435-2449. https://doi.org/10.1074/mcp.0113.037135.
(35) Bettinger, J. Q.; Welle, K. A.; Hryhorenko, J. R.; Ghaemmaghami, S. Quantitative Analysis of in Vivo Methionine Oxidation of the Human Proteome. J. Proteome Res. 2020, 19 (2), 624-633. https://doi.org/10.1021/acs.jproteome.9b00505.
(36) Bryan, C. M.; Rocklin, G. J.; Bick, M. J.; Ford, A.; Majri-Morrison, S.; Kroll, A. V.; Miller, C. J.; Carter, L.; Goreshnik, I.; Kang, A.; DiMaio, F.; Tarbell, K. V.; Baker, D. Computational Design of a Synthetic PD-1 Agonist. Proc. Natl. Acad. Sci. U.S.A. 2021, 118 (29), c2102164118. https://doi.org/10.1073/pnas.2102164118.
(37) Helou, D. G.; Shafici-Jahani, P.; Lo, R.; Howard, E.; Hurrell, B. P.; Galle-Treger, L.; Painter, J. D.; Lewis, G.; Soroosh, P.; Sharpe, A. H.; Akbari, O. PD-1 Pathway Regulates ILC2 Metabolism and PD-1 Agonist Treatment Ameliorates Airway Hyperreactivity. Nat Commun 2020, 11 (1), 3998. https://doi.org/10.1038/s41467-020-17813-1.
(38) Curnock, A. P.; Bossi, G.; Kumaran, J.; Bawden, L. J.; Figueiredo, R.; Tawar, R.; Wiseman, K.; Henderson, E.; Hoong, S. J.; Gonzalez, V.; Ghadbane, H.; Knight, D. E. O.; O'Dwyer, R.; Overton, D. X.; Lucato, C. M.; Smith, N. M. G.; Reis, C. R.; Page, K.; Whaley, L. M.; McCully, M. L.; Hearty, S.; Mahon, T. M.; Weber, P. Cell-Targeted PD-1 Agonists That Mimic PD-L1 Are Potent T Cell Inhibitors. JCI Insight 2021, 6 (20), e152468. https://doi.org/10.1172/jci.insight.152468.
(39) Song, M.-Y.; Hong, C.-P.; Park, S. J.; Kim, J.-H.; Yang, B.-G.; Park, Y.; Kim, S. W.; Kim, K. S.; Lec, J. Y.; Lec, S.-W.; Jang, M. H.; Sung, Y.-C. Protective Effects of Fc-Fused PD-L1 on Two Different Animal Models of Colitis. Gut 2015, 64 (2), 260-271. https://doi.org/10.1136/gutjnl-2014-307311.
(40) Ryman, J. T.; Meibohm, B. Pharmacokinetics of Monoclonal Antibodies: Pharmacokinetics of Monoclonal Antibodies. CPT Pharmacometrics Syst. Pharmacol. 2017, 6 (9), 576-588. https://doi.org/10.1002/psp4.12224.
(41) Huang, D.; Wen, W.; Liu, X.; Li, Y.; Zhang, J. Z. H. Computational Analysis of Hot Spots and Binding Mechanism in the PD-1/PD-L1 Interaction. RSC Adv. 2019, 9 (26), 14944-14956. https://doi.org/10.1039/C₉RA01369E.
(42) Fujiwara, K.; Toda, H.; Ikeguchi, M. Dependence of Alpha-Helical and Beta-Sheet Amino Acid Propensities on the Overall Protein Fold Type. BMC Struct Biol 2012, 12 (1), 18. https://doi.org/10.1186/1472-6807-12-18.

1. A composition comprising a peptide having SEQ ID NO. 5 and its variant thereof, wherein a variant peptide has at least 85% homologous to SEQ ID No. 5, wherein the variant peptide does not contain mutation at tyrosine 12 residue of SEQ ID NO. 5.

2. The composition of claim 1, wherein the peptide having SEQ ID NO. 5 or the variant thereof binds with molecules of programmed death protein-1 (PD-1) to form a complex that modulate release of an inflammatory cytokine from a cell.

3. The composition of claim 1, wherein the peptide having SEQ ID NO. 5 or the variant thereof is a PD-1 agonist used to treat an inflammatory disease.

4. The composition of claim 1, wherein a variant peptide of SEQ ID NO. 5 comprises one or more amino acids chemically modified to provide protease resistance to the variant peptide compared to SEQ ID NO. 5 and/or increase stability of the variant peptide compared to SEQ ID NO. 5.

5. The composition of claim 4, wherein a chemical modification comprises methylation of an amino acid.

6. The composition of claim 1, wherein a variant peptide of SEQ ID NO. 5 does not contain substitution of methionine with norleucine.

7. The composition of claim 4, wherein the variant peptide is cyclized.

8. (canceled)

9. The composition of claim 4, wherein the chemical modification comprises one or more amino acids comprising a branch forming compound comprising an olefin-terminated amino acid to increase stability or maintain beta hairpin structure of the variant peptide.

10. (canceled)

11. The composition of claim 1, wherein the variant peptide comprises substitution of one or more amino acids with non-natural amino acids comprising D-amino acids or fluorine analog.

12. The composition of claim 1, wherein the variant peptide of SEQ ID NO. 5 forms a covalent or noncovalent bond with polyethylene glycol (PEG) molecule.

13. The composition of claim 4, wherein the chemical modification comprises isosteres of the peptide bond connecting one or more amino acids; wherein the scaffolding unit comprises a modified or unmodified semi-rigid nucleic acid polymer.

14. The composition of claim 4, wherein the variant peptide of SEQ ID NO. 5 is attached to a scaffolding unit via a chemical linker.

15. The composition of claim 14, wherein the chemical linker comprises azido group.

16-18. (canceled)

19. The composition of claim 1, wherein the peptide having SEQ ID NO. 5, and the variant thereof has a serum stability of at least 6 hours.

20. (canceled)

21. The composition of claim 120, wherein the peptide having SEQ ID NO. 5, and the variant thereof degrades less than 30% in the serum for 48 hours.

22. (canceled)

23. The composition of claim 2, wherein the peptide having SEQ ID NO. 5, and the variant thereof decreases release of an inflammatory cytokine in a dose-dependent manner.

24. The composition of claim 4, wherein the variant peptide of SEQ ID NO. 5 comprises one or more residues that allow formation of a disulfide or thioether bond.

25. The composition of claim 24, wherein the variant peptide comprises SEQ ID NO. 9, SEQ ID NO. 10, SEQ ID NO. 11, SEQ ID NO. 12 or 13.

26-28. (canceled)

29. The composition of claim 4, wherein the variant peptide does not contain mutation at 2, 4, 11, 13, or 14 residues of SEQ ID NO. 5.

30-31. (canceled)

32. The composition of claim 29, wherein the variant peptide does not contain mutation at 2, 4, and 11-14 residues of SEQ ID NO. 5.

33-34. (canceled)


SEQUENCE LISTING (14 sequences)

SEQ ID NO: 1 - protein, 17 aa, Homo sapiens
YRCMISYGGADYKRITV

SEQ ID NO: 2 - protein, 17 aa, synthetic construct
YRCMISYGGADYKRITV

SEQ ID NO: 3 - protein, 17 aa, synthetic construct
YRCMISNGGADYKRITV

SEQ ID NO: 4 - protein, 19 aa, synthetic construct
  DISULFID 1..19: note = Intra chain disulphide
CYRAMISYGGADYKRITVC

SEQ ID NO: 5 - protein, 16 aa, synthetic construct
  SITE 14: note = N-methyl Arginine
  DISULFID 1..16: note = intra chain disulphide formation
CRAMISYGGADYKXIC

SEQ ID NO: 6 - protein, 16 aa, synthetic construct
  SITE 4: note = Nle
  DISULFID 1..16: note = Intra chain disulphide formation
  SITE 14: note = N-methyl arginine
CRAXISYGGADYKXIC

SEQ ID NO: 7 - protein, 16 aa, Synthetic construct
  DISULFID 1..16: note = intra chain disulphide formation
CRAMISYKGADYKRIC

SEQ ID NO: 8 - protein, 16 aa, Synthetic construct
  DISULFID 1..16: note = intra chain disulphide formation
CRALIAYKGADYKRIC

SEQ ID NO: 9 - protein, 16 aa, Synthetic construct
  SITE 14: note = N-methyl arginine
  CROSSLNK 1..16: note = Cys..alanine thioether bond
ARAMISYGGADYKXIC

SEQ ID NO: 10 - protein, 16 aa, synthetic construct
  SITE 14: note = N-methyl arginine
CRAMISYGGADYKXIC

SEQ ID NO: 11 - protein, 17 aa, synthetic construct
  SITE 1: note = K(N3)
  SITE 15: note = N-methyl arginine
XCRAMISYGGADYKXIC

SEQ ID NO: 12 - protein, 17 aa, synthetic construct
  SITE 1: note = azido-lysine residue
  SITE 4: note = olefin-terminated alanine
  SITE 15: note = N-methyl arginine
  SITE 16: note = non-natural amino acid
XCRXMISYGGADYKXXC

SEQ ID NO: 13 - protein, 17 aa, synthetic construct
  SITE 1: note = azido-lysine residue
  SITE 7: note = olefin-terminated amino acid
  SITE 11: note = olefin-terminated non natural amino acid
  SITE 15: note = N-methyl arginine
XCRAMIXYGGXDYKXIC

SEQ ID NO: 14 - protein, 17 aa, synthetic construct
YRCMISYGGADEKRITV